(19) United States
(12) Patent Application Publication
(10) Pub. No.: US 2009/0257968 A1
(43) Pub. Date: Oct. 15, 2009

(54) Gels containing anionic surfactants and coupling agents

(76) Inventors: Richard Walton, Monroe, NC (US); Edward T. Borish, Westerville, OH (US)

Correspondence Address: Isaac A. Angres, Suite 301, 2001 Jefferson Davis Highway, Arlington, VA, 22202, US

(21) Appl. No.: 12/453,308

(22) Filed: May 6, 2009

Related U.S. Application Data
(63) Continuation-in-part of application No. 11/087,570, filed on Mar. 24, 2005.
(60) Provisional application No. 60/556,052, filed on Mar. 25, 2004.

(51) Int. Cl. A61K 47/18; B01F 17/16; A61K 47/12; A61Q 1/06; A61Q 1/04; C11D 17/00; A61K 36/886; A61K 33/08; A61K 33/24; A61K 33/22; A61K 33/26; A61K 31/718; A61K 31/765; A61K 31/047; A61K 31/56; A61K 36/06; A61K 31/415; A61K 35/60; A61K 31/235; A61K 31/045; A61K 33/10; A61K 33/30; A61K 33/04

(52) U.S. Cl. 424/64; 516/204; 516/203; 510/152; 514/785; 514/788; 514/772; 424/744; 424/682; 424/653; 424/659; 424/647; 424/776; 514/60; 424/78.03; 514/738; 514/170; 424/722; 424/195.16; 514/389; 424/554; 514/543; 514/724; 424/717; 424/715; 424/642; 424/600

(57) ABSTRACT

The present invention provides an enhanced gelling agent composition comprising a mixture of an anionic surfactant and a coupling agent. The invention also describes compositions in gel form comprising: (a) a solvent selected from the group consisting of polar solvents, non-polar solvents and combinations thereof; and (b) a gelling agent in an amount sufficient to impart gelation to the composition, the gelling agent being a mixture comprising an anionic surfactant and a coupling agent. The gels of the invention may be substantially anhydrous or may optionally contain water as part of the formulation.

This application is a continuation-in-part of U.S. patent application Ser. No. 11/087,570 filed Mar. 24^{th}, 2005, the entire contents of which are incorporated by reference herein. This application also claims the priority benefit under 35 U.S.C. section 119 of U.S. provisional application Ser. No. 60/556,052 entitled “Anhydrous gels containing metallic soaps and coupling agents” filed Mar. 25, 2004, which is in its entirety herein incorporated by reference.

### FIELD OF INVENTION

This invention generally relates to gelled-oil compositions. More particularly, the invention relates to gelled-oil compositions which include metallic soaps and coupling agents.

The invention further relates to the gelation of a substrate with a metallic soap and a coupling agent. The present invention also describes anhydrous and non-anhydrous gel compositions comprising a substrate such as a mineral oil and a gelling agent comprising a metallic soap and a coupling agent. The invention further relates to methods of making gel compositions.

The present invention also relates to the gellation or solidification of polar and non-polar organic liquids as well as combinations thereof, and to products obtained therefrom.

The invention also refers to a new gel product which contains oils or fats and suitable gel forming agents and which has improved characteristics as regards to stability and structure.

This invention is also directed to cleansers i.e., facial cleansers wherein mineral oil is the principal active ingredient, and more specifically to gels of mineral oils.

The instant invention further relates to compositions useful as carriers for pharmaceutical actives such as antiseptics, antifungals, sunscreens, deodorants and the like; and more particularly, to such pharmaceutical compositions in the form of a gel or gel stick.

This invention also involves a new pharmaceutical vehicle for active substances in the form of an anhydrous and non-anhydrous gel, namely for substances which are sensitive to oxidation. The invention also provides a new cosmetic vehicle in the form of an anhydrous and non-anhydrous gel, namely for use in deodorants, antiperspirants, topical formulations and all other cosmetic uses.

Additionally, the present invention relates to a solid composition, for example a cosmetic composition such as a care, treatment and/or make-up composition for the skin, including the scalp, and/or for the lips of human beings, comprising a thickened liquid phase. The composition can be in the form of a stick or tube of make-up, such as a lipstick.

The gels of the present invention can be used in many cosmetic and topical medicinal applications. Also, the gels of the invention are applicable for making candles and air fresheners.

### BACKGROUND OF THE INVENTION

It is traditional practice to use a structured, i.e., thickened or gelled, liquid phase in compositions, such as in cosmetic and dermatological compositions, in order to obtain the desired consistency. The thickening of organic liquids such as mineral oils (or of phases that are liquid at room temperature) makes it easier to take up the product from its packaging without any significant loss, to limit the diffusion of the product to the local treatment area, to distribute the product uniformly over the local treatment area, or to be able to use the product in amounts that are sufficient to obtain the desired cosmetic or dermatological effect. This is especially the case in solid compositions such as deodorants, lip balms and lipsticks, concealer products and cast-foundations. This thickening is desirable for personal care, hygiene or make-up compositions such as lipsticks, which are preferably distributed homogeneously over the local surface to be treated, as well as for hair compositions, which are preferably spread and distributed uniformly along the keratin fibers and which preferably do not run down the forehead, the nape of the neck, the face or into the eyes.

To overcome the above problems, use is usually made of waxes or fillers. Unfortunately, these waxes and/or fillers have a tendency to make the composition matte and opaque, which is not always desirable, in particular for a lipstick. Specifically, women are always in search of a lipstick in the form of a tube which gives a glossy film; moreover, certain compositions such as lip balms or ointments can be in the form of translucent, or even transparent, sticks.

It is also customary practice to thicken oils with polymeric thickeners. Unfortunately, the known thickeners for oils have to be used in large amounts in order to obtain a gel of high viscosity, for example of greater than 1.3 Pa.s. However, too large an amount of thickener can give the composition inadequate cosmetic properties, in particular, a sticky feel and a lack of slipperiness. These drawbacks can potentially be very inconvenient, or even prohibitive.

The structuring of the liquid phase makes it possible to limit its exudation from solid compositions and, in addition, to limit the migration of this phase in wrinkles and fine lines after it has been deposited on the skin or the lips, which is a particularly desired quality for a lipstick. The reason for this is that a large migration of the liquid phase, charged with dyestuffs, leads to an unaesthetic effect around the lips, which particularly accentuates wrinkles and fine lines. This migration is often mentioned by women as a major defect of conventional lipsticks.

Also, in the practical use of the above organic liquids, it is often desirable to render them solid. If a container is damaged or broken open, the organic liquids stored therein flow out and spread over a wide area, and recovery of the spilled liquid is difficult. If the spilled organic liquids are inflammable, they burn once ignited. For example, fire and smoke inhalation cause many deaths in otherwise survivable aircraft accidents. The fires usually are caused when the highly volatile fuel spills from damaged tanks and splatters over a wide area. Fuel vaporizes and is easily ignited by hot engine parts or sparks from metal impact. On the contrary, when fuels are gelled, the degree of vaporization and the extent to which the fuel is scattered upon impact are decreased, and so the danger of rapidly spreading fire or explosion is substantially reduced. Moreover, gelling agents for organic liquids are useful for facilitating the removal or recovery of spilled polar and non-polar organic liquids or other aggregates of polar and non-polar organic liquids, and for preventing the leakage or spillage of such polar and non-polar organic liquids from leaking tanks or holes.

Gelling agents and thickeners are generally regarded as substances added during the manufacturing process to achieve a desired consistency or viscosity. Gelling agents are, typically, added as fluidity modifiers or solidifiers for paints and inks, gelling agents for recovering spilt oils, solidifiers for pesticide formulations, anticlumping agents for painting or adhesive materials, processing aids for macromolecules as well as gelling agents or solidifiers for perfumes or other cosmetic compositions. Traditional gelling agents include metal salts of stearic acid, clays, pectins, carrageenan, alginates, nylon, fumed silicas, organic derivatives of castor oil and ethoxylated saturated fatty acids. More recent gelling agents include dibenzylidenesorbitol and derivatives thereof having a substituent or substituents on the aromatic ring, 12-hydroxystearic acid, acylated amino acid amides and cholesterol derivatives.

While the prior art discloses a wide variety of useful gelling agents, there is still a need for additional gelling agents which provide improved gel stability and/or hardness at reduced manufacturing costs. The present inventor has found that gelling agents in the form of a mixture of a metallic soap and a coupling agent provides enhanced gel stability and/or hardness.

The shortcomings of the prior art gelling agents noted above may be overcome by employing the enhanced gelling mixtures in accordance with the present invention.

### OBJECTS OF THE INVENTION

Accordingly, it is an object of the present invention to provide improved gelling agents.

Another object of the present invention is to provide improved gelling agents for formulating stable gel or stick compositions.

It is a further object of the instant invention to produce a composition, such as a cosmetic composition, which is in solid form, and is capable of conserving good cosmetic properties, such as a certain level of translucency.

A still further object of the invention is to provide a composition, such as a cosmetic or dermatological composition, which is in solid form, comprising at least one organic liquid and a gelling agent containing a metallic soap and a coupling agent.

These and other objects of the present invention will more readily become apparent from the description and examples which follow.

### SUMMARY OF THE INVENTION

The present invention provides a composition suitable for gelling polar, non-polar solvents (by solvent applicant means a liquid whose viscosity can be increased) and combinations thereof comprising: (a) a metallic soap; and (b) an enhancer comprising a coupling agent.

The instant invention also provides a composition suitable for gelling polar, non-polar solvents and combinations thereof comprising: (a) a metallic soap; and (b) an enhancer selected from the group consisting of: isocetyl citrate, isoarachidyl citrate, POE(5) diTMP tetra-isostearate, trimethyl soya quaternary ammonium chloride, (C₁₂₋₁₈)dialkyldimethyl-ammonium chloride, soybean oil DEA transester, castor oil, POE(4) cetyl stearyl alcohol, POE(4) decyl alcohol, POE(6) decyl alcohol, POE(2) 2-ethylhexyl alcohol, Epal 1214 phosphate, C₈₋₁₀ triglyceride, CFA/DEA transamide TOFA salt, PPG 2025 TOFA ester, POE(2.5)cocoamine DES quaternary, stearic acid DEA amide, glycerine sesqui-cocoate, TOFA/DEA amide/soap, POE(3.3) CFA/MEA amide, DMSD/TEA DES quaternary, glycerine sesqui-C₈₋₁₀, POE(2) cocoamine, POE(5) castor oil, CFA/DEA transamide, PEG 600 dilaurate, PEG 200 dilaurate, PEG 400 dilaurate, PEG 600 ditallate, PEG 400 ditallate, POE(5) hydrogenated castor oil, POE(15) hydrogenated tallow amine DES quaternary, POE(4) laurate, POE(4) tallate, POE(2) tallow amine, POE(2) tallow amine DES quaternary, sorbitan monostearate (SMS), glycerine dilaurate, glycerine monosoyate, glycerine monostearate (GMS), hydrogenated castor oil (HCO), PEG 200, PEG 400, POE(1.1) laurate, POP(1.1) laurate, sorbitan monolaurate (SML), sorbitan monooleate (SMO), sorbitan monopalmitate (SMP), sorbitan trioleate (STO), soybean oil, triglycerol monostearate, decaglycerol monostearate, and tri-C₁₂₋₁₄ borate ester.

The invention is also directed to a composition in gel form comprising: (a) an anhydrous or non-anhydrous liquid carrier; and (b) a gelling agent in an amount sufficient to impart gelation to the composition, the gelling agent being a mixture comprising a metallic soap and a coupling agent, wherein the composition is substantially anhydrous or optionally may contain some water.

The instant invention further describes a composition in gel form comprising: (a) a solvent selected from the group consisting of polar, non-polar solvents and combinations thereof; and (b) a gelling agent in an amount sufficient to impart gelation to the composition, the gelling agent being a mixture comprising a metallic soap and a coupling agent ester, wherein the composition is substantially anhydrous or optionally may contain some water.

The present invention also relates to a cosmetic gel product on the basis of oils and gelling agents which product comprises 5 to 99% by weight of a substrate selected from the group consisting of: oils, mineral oils, hydrogenated hydrocarbons, alkenes, terpenes, mono esters, di esters, tri esters and mixtures thereof, 1 to 95% by weight of a gelling agent comprising a mixture of a metallic soap and a coupling agent and all percentages being relative to the weight of the gel product, and wherein the gel product is transparent or a translucent solid stick.

The instant invention is also directed to a gel comprising: (a) a gelling agent comprising a mixture of a metallic soap and a glyceryl ester; and (b) an anhydrous liquid carrier.

The invention also provides a process for preparing a gel, which comprises: homogeneously admixing an anhydrous or non-anhydrous organic liquid with an effective gel forming amount of a composition comprising a metallic soap and a glyceryl ester; and allowing the mixture to stand until gel formation occurs.

### DESCRIPTION OF THE PREFERRED EMBODIMENTS

In its broader aspects, the gelling agent compositions of the invention comprise 1% to 99% by weight of a surfactant and 99% to 1% by weight of the enhancer or coupling agent. The ratio of the two components may be adjusted as desired to achieve the desired gel consistency required by the formulator.

Additionally, the gelling agent compositions of the invention comprise 1% to 99% by weight of an anionic surfactant and 99% to 1% by weight of the enhancer or coupling agent. The ratio of the two components may be adjusted as desired to achieve the desired gel consistency required by the formulator.

This invention is also directed to gelling compositions and the gelation of a substrate with a metallic soap and a coupling agent. The substrate can be hydrocarbons (mineral oil, kerosene, diesel fuel, terpenes, etc.), glycerides (soybean oil, castor oil, synthetic mono-, di-, and tri-glycerides, etc.), alcohols (ethanol, isopropanol, etc), or various materials (toluene, ethylene glycol, ethyl acetate, water, etc.) and silicones. The metallic soap is used to achieve gelation of the substrate. However, in nonpolar systems, the metallic soap is either insoluble and as such cannot provide gelation, or soluble and provides a gel structure that does not hold the substrate inclusive. The coupling agents of the invention solubilize the metallic soap into the substrate and provides a synergistic force that causes a gel to form. The resulting gels of the invention are essentially free from surface liquid.

The gelling agents of the invention are enhanced mixtures of an inorganic gelling agent or a metallic soap with an enhancer which is an organic material having ester moieties or alkylene oxide moieties, as well as amine and alkylene oxide moieties. Salts of C₁₋₅ alcohols may also be used instead of metallic soaps. The gelling agent compositions of the invention comprise 1% to 99% by weight of a metallic soap and 99% to 1% by weight of the enhancer or coupling agent. The ratio of the two components may be adjusted as desired to achieve the desired gel consistency required by the formulator. The preferred metallic soap component for use in the anhydrous gel compositions of the present invention are salts of fatty acids, wherein the fatty acid moiety has from about 12 to about 40 carbon atoms, preferably from about 12 to about 22 carbon atoms, more preferably from about 16 to about 20 carbon atoms, most preferably about 18 carbon atoms. Non-limiting examples of fatty acids suitable for making the fatty acid gellants include acids such as myristic, palmitic, stearic, oleic, lauric, arachidic, behenic, linoleic, linolenic, margaric and combinations thereof. These fatty acids are preferably derived from sources such as coconut oil, beef tallow, lanolin, fish oil, beeswax, palm oil, castor oil, peanut oil, olive oil, castor oil, cottonseed oil, soybean oil, corn oil, rapeseed oil, rosin acids, greases, and other natural sources, or are derived by synthetic or semisynthetic methods well known to those skilled in the formulation art. Suitable salt forming cations for use with these gelling agents include metal salts such as alkali metals, e.g. sodium and potassium, and alkaline earth metals, e.g. magnesium, and aluminum. Preferred are sodium and potassium salts, more preferably sodium stearate, sodium palmitate, sodium laurate, sodium arachidate, sodium behenate, potassium stearate, potassium palmitate, sodium myristate, aluminum monostearate, and combinations thereof. Most preferred is sodium stearate. The preferred metal salts are those derived from alkali metals and alkaline earth metals. Other metals such as aluminum and zinc are also usable in making the metallic soap gellants of the invention. Exemplary gelling agents may be selected from the group consisting of: aluminum isopropoxide, aluminum stearate, borax, calcium acetate, calcium stearate, dipotassium dodecenyl succinate, disodium dimerate, lithium stearate, magnesium stearate, potassium acetate, potassium stearate, sodium 12-hydroxystearate, sodium 2-ethylhexanoate, sodium azelate, sodium carbonate, sodium dodecyl sulfate, sodium EDTA, tetra sodium EDTA, sodium oleate, sodium oxalate, sodium stearate, stearic acid and zinc stearate.

Additional soap components that are intended to be included as part of this invention include other anionic materials such as the alkali metal salts, alkaline earth metal salts, ammonium salts and salts of amines substituted with organic groups, such as monoethanolamine salts, diethanolamine salts, triethanolamine salts and 2-amino-2-methylpropane-1,3-diol salts, of alkyl sulfates having 8-22 carbon atoms in the alkyl group, polyoxyethylene (1-10 moles) alkyl ether sulfates having 8 to 22 carbon in the alkyl group, polyoxyethylene (1-10 moles) alkylphenyl ether sulfates having 8 to 12 carbon atoms in the alkyl group, olefin sulfonates having 8-22 carbon atoms and alkylbenzene sulfonates having a straight or branched alkyl group of 9 to 18 carbon atoms, preferably 12 carbon atoms, on the average.

Exemplary of useful anionic materials are ammonium lauryl sulfate, sodium lauryl diethoxy sulfate, ammonium lauryl triethoxy sulfate, sodium alpha C₁₆ olefin sulfonate, sodium C₁₄ paraffin sulfonate, sodium coco monoglyceride sulfate, triethanolamine cetyl sulfate and dimethylamine myristyl sulfate. However, for best results it is preferred to utilize higher alkyl sulfate, higher alkyl poly-lower alkoxy sulfate or a mixture of such higher alkyl sulfate and such higher alkyl ether sulfate, such as lauryl sulfate and lauryl diethoxy sulfate or lauryl triethoxy sulfate, with either being present in greater or equal proportion and in ammonium, triethanolamine and/or sodium salt form.

Suitable anionic surfactants in addition to the alkyl sulphates, alkyl ether sulphates, and alkaryl sulphonates include the alkanoyl isethionates, alkyl succinates, alkyl sulphosuccinates, N-alkoyl sarcosinates, alkyl phosphates, alkyl ether phosphates, alkyl ether carboxylates, and alpha-olefin sulphonates, especially their sodium, magnesium ammonium and mono-, di- and triethanolamine salts. The alkyl and acyl groups generally contain from 8 to 18 carbon atoms and may be unsaturated.

Examples of other suitable anionic surfactants include sodium oleyl succinate, ammonium lauryl sulphosuccinate, sodium dodecylbenzene sulphonate, triethanolamine dodecylbenzene sulphonate, sodium cocoyl isethionate, sodium lauroyl isethionate and sodium N-lauryl sarcosinate. As stated above, the most preferred anionic surfactants are sodium lauryl sulphate, triethanolamine lauryl sulphate, triethanolamine monolauryl phosphate, sodium lauryl ether sulphate 1EO, 2EO and 3EO, ammonium lauryl sulphate and ammonium lauryl ether sulphate 1EO, 2EO and 3EO.

Other suitable gellants suitable for use in the anhydrous gel compositions include hydroxy acids, fatty acids, esters and amides of fatty acids and fatty acid salts, hydroxy fatty acids, cholesterolic materials, lanolinolic materials, and other gellants known for use as gelling agents or which are otherwise described in detail hereinafter.

The second component of the enhanced gelling agents of invention is a coupling agent selected from the group consisting of:

(1) Monoester alkoxylates having the following chemical structure

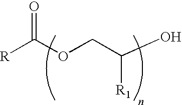

wherein R═H, C₁₋₂₅ primary alkyl, secondary alkyl, tertiary alkyl, branched alkyl, C₂-C₂₅ alkenyl, C₂-C₂₅ alkynyl, C₆-C₁₅ substituted alkylaryl, saturated and unsaturated C₅-C₁₀ cycloaliphatic wherein all Rs' may be optionally substituted with halogen or other functional groups selected from the group consisting of hydroxy, carboxy, sulfonate, aldehyde, ketone, R₁═H, C₁₋₂, phenyl and n=1-200. Particular compounds which are included within the above generic structure include POP(1.1) laurate, POE(1.1) laurate, POP(5) laurate, POE(1) Tung oil fatty acid, Ethox MO-5, Ethox ML-5, Ethox MI-9 and Ethox 2610.

(2) Monoamide derivatives having the following structural chemical formula

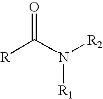

wherein R═H, C₁₋₂₅ primary alkyl, secondary alkyl, tertiary alkyl, branched alkyl, C₂-C₂₅ alkenyl, C₂-C₂₅ alkynyl, C₆-C₁₅ substituted alkylaryl, saturated and unsaturated C₅-C₁₀ cycloaliphatic wherein all Rs' may be optionally substituted with halogen or other functional groups selected from the group consisting of hydroxy, carboxy, sulfonate, aldehyde, ketone, R₁═H, C₁₋₂₀, (—CH₂CHR₃O—)ₙH (n=1-200) R₃═H, C₁₋₂, phenyl, R₂═H, C₁₋₂₀, —(CH₂CHR₃O—)ₙ (n=1-200) R₃═H, C₁₋₂, phenyl. Representative examples of compounds within the scope of the above formula include Ethox COA, ERS 00209, Ethox 3166, Amide PF, Ethox 2984, Ethox 2449 and Isostearic DEA.

(3) Diamides having the following chemical structure

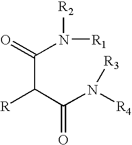

wherein R═H, C₁₋₂₅ primary alkyl, secondary alkyl, tertiary alkyl, branched alkyl, C₂-C₂₅ alkenyl, C₂-C₂₅ alkynyl, C₆-C₁₅ substituted alkylaryl, saturated and unsaturated C₅-C₁₀ cycloaliphatic wherein all Rs' may be optionally substituted with halogen or other functional groups selected from the group consisting of hydroxy, carboxy, sulfonate, aldehyde, ketone, R₁, R₂, R₃, and R₄═H, C₁₋₂₀, —(CH₂CHR₅O—)ₙH (n=1-200) R₅═H, C₁₋₂, phenyl, and R₁, R₂, R₃, and R₄ may all be the same or different at each designated position. Typical compounds that are included within the scope of the above chemical structure include MEA octenyl succinate, DEA/MEA octenyl succinate stearate, DEA octenyl succinate stearate and MEA octenyl succinate stearate.

(4) Alcohol alkoxylates having the following chemical structure

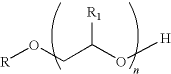

wherein R═H, C₁₋₂₅ primary alkyl, secondary alkyl, tertiary alkyl, branched alkyl, C₂-C₂₅ alkenyl, C₂-C₂₅ alkynyl, C₆-C₁₅ substituted alkylaryl, saturated and unsaturated C₅-C₁₀ cycloaliphatic wherein all Rs' may be optionally substituted with halogen or other functional groups selected from the group consisting of hydroxy, carboxy, sulfonate, aldehyde, ketone, R₁═H, C₁₋₂, Phenyl, n=1-200. Compounds that are within the scope of the above formula include Ethal EH-2, Ethal DA-6, Ethal DA-4, Ethal 368, 2-Phenoxyethanol, POE(2) BPA, Ethox BA-25, POE(7) decyl alcohol, POE(3) TMP, Ethal LA-4, Ethal LA-23, Ethal 326, POP(200) I-24, and POP(2.6) Epal 1214.

(5) Triglycerides (natural and synthetic) having the following chemical structure:

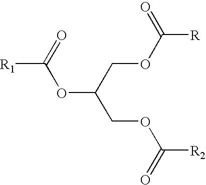

wherein R, R₁, and R₂═H, C₁₋₂₅ primary alkyl, secondary alkyl, tertiary alkyl, branched alkyl, C₂-C₂₅ alkenyl, C₂-C₂₅ alkynyl, C₆-C₁₅ substituted alkylaryl, saturated and unsaturated C₅-C₁₀ cycloaliphatic wherein all Rs' may be optionally substituted with halogen or other functional groups selected from the group consisting of hydroxy, carboxy, sulfonate, aldehyde, ketone, and may be the same or different at each position. Typical examples include soybean oil, hydrogenated castor oil, castor oil, and Ethox 2156.

(6) Glycerides having the following chemical structure:

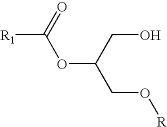

wherein R₁═H, C₁₋₂₅ primary alkyl, secondary alkyl, tertiary alkyl, branched alkyl, C₂-C₂₅ alkenyl, C₂-C₂₅ alkynyl, C₆-C₁₅ substituted alkylaryl, saturated and unsaturated C₅-C₁₀ cycloaliphatic wherein all R₁s' may be optionally substituted with halogen or other functional groups selected from the group consisting of hydroxy, carboxy, sulfonate, aldehyde, ketone, R═H or C₁₋₂₅ alkyl carbonyl, and may be aromatic, unsaturated, saturated, branched, linear, cyclic, etc. Compounds that can be used and that are within the scope of the above structural formula include glycerol sesquibenzoate, glycerol sesqui-2-ethylhexanoate, glycerol monostearate, glycerol monosoyate, glycerol dilaurate, Ethox 4076, Ethox 4075, Ethox 4074, Ethox 4073, Ethox 3051, glycerol dimyristate, glycerol distearate, Glycerol monoisostearate.

(7) Sorbitan esters having the following chemical structure:

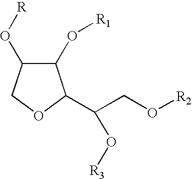

wherein each substituent R, R₁, R₂, and R₃ is independently selected from H, C₁-C₂₅ alkyl, alkylcarbonyl or alkenylcarbonyl, or —(CH₂CHR₄O—)ₙR′ (n=1-200), wherein R′ is selected from the group consisting of H, C₁-C₂₅ alkyl, alkylcarbonyl or alkenyl carbonyl, R₄═H, C₁₋₂, phenyl. R, R₁, R₂, and R₃ may be optionally substituted with halogen or other functional groups selected from the group consisting of hydroxy, carboxy, sulfonate, aldehyde, ketone, and R, R₁, R₂, and R₃ may be the same or different at each position. Typical examples include sorbitan monolaurate, sorbitan monooleate, sorbitan monopalmitate, sorbitan trioleate and Ethsorb S.

(8) Polyglycerols having the following chemical formula:

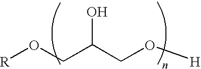

wherein R═H, C₁₋₂₅ alkyl, alkylcarbonyl or alkenylcarbonyl, R may be optionally substituted with halogen or other functional groups selected from the group consisting of hydroxy, carboxy, sulfonate, aldehyde, ketone, and n=2-25. Typical compounds representative of this class includes Polyglycerol-3 stearate, and Polyglycerol-8 stearate.

(9) Polyalkoxylates having the following chemical structure:

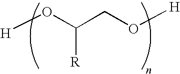

wherein R═H, C₁₋₂₅ primary alkyl, secondary alkyl, tertiary alkyl, branched alkyl, C₂-C₂₅ alkenyl, C₂-C₂₅ alkynyl, phenyl, C₆-C₁₅ substituted alkylaryl, saturated and unsaturated C₅-C₁₀ cycloaliphatic wherein all Rs' may be optionally substituted with halogen or other functional groups selected from the group consisting of hydroxy, carboxy, sulfonate, aldehyde, ketone and n=1-200. Suitable compounds within the scope of the above structural formula include PEG 200, PEG 400, ethylene glycol, and PPG 460.

(10) Ethoxylated triglycerides (castor oil derivatives) of the following structural formula

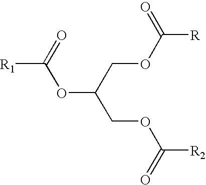

wherein R=alkoxylated ricinoleic residue, or alkoxylated 12-hydroxystearic acid residue and R, R₁, and R₂ may be the same or different at each position. Typical examples include Ethox HCO-5 and Ethox CO-5.

(11) Sorbitol alkoxylate esters of the following chemical formula:

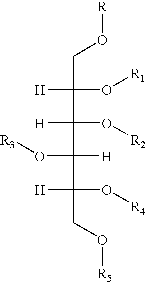

wherein each substituent R, R₁, R₂, R₃, R₄ and R₅ is independently selected from H, C₁-C₂₅ alkyl, alkylcarbonyl or alkenylcarbonyl, or —(CH₂CHR₄O—)ₙR′ (n=1-200), wherein R′ is selected from the group consisting of H, C₁-C₂₅ alkyl or alkenyl carbonyl, R₄═H, C₁₋₂, phenyl and R, R₁, R₂, and R₃ may be optionally substituted with halogen or other functional groups selected from the group consisting of hydroxy, carboxy, sulfonate, aldehyde, ketone, and R₁, R₂, R₃, R₄, and R₅ may be the same or different at each position. Examples of the above formula include ethoxylated sorbitol tetraoleate and ethoxylated sorbitol hexaoleate.

(12) Sulfates having the following chemical formula:

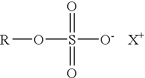

wherein R═H, C₁₋₂₅ alkyl or alkenyl, or —(CH₂CHR₄O—)ₙR′ (n=1-200), R′ is C₁-C₂₅ alkyl or alkenyl carbonyl, R₄═H, C₁₋₂, phenyl, and may be aromatic, unsaturated, saturated, branched, linear or cyclic group, X⁺=alkali or alkaline earth metal or ammonium.

(13) Polyamines having the following structural formula:

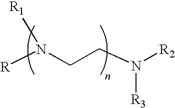

wherein R, R₁, R₂ and R₃ are selected from the group consisting of R═H, C₁₋₂₅, or —(CH₂CHR₄O—)ₙH (n=1-200) R₄═H, C₁₋₂, phenyl, and may be aromatic, unsaturated, saturated, branched, linear, cyclic, and R, R₁, R₂, and R₃ may be the same or different and n=1-25. Examples of compounds within the scope of the above formula include EDTA, DETA, POE(3) DETA, and POE(10) DETA.

(14) Citrate esters having the following chemical formula:

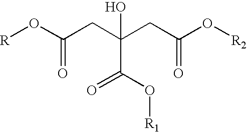

wherein R═H, C₁₋₂₅, or —(CH₂CHR₄O—)ₙR′ (n=1-200), R′ is C₁-C₂₅ alkyl or alkenyl carbonyl R₄═H, C₁₋₂, phenyl, and may be aromatic, unsaturated, saturated, branched, linear or cyclic hydrocarbon group. Typical compounds that are useful in this family include ERS 00204, ERS 00205, Octyl/decyl sesquicitrate and Octyl/decyl sesquicitrate Na+.

(15) Alkylene oxide esters having the following formula:

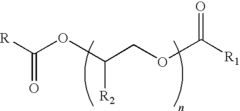

wherein R, R₁═H, C₁₋₂₅, and may be C₆-C₁₀ aromatic, or unsaturated, saturated, branched, linear or cyclic hydrocarbon groups having up to 25 carbon atoms, and R and R₁ may be the same or different, R₂═C₁₋₂, phenyl, n=1-200. Examples of the above formula include Ethox DO-9, Ethox DO-14, Ethox DL-5, Ethox DL-9, Ethox DL-14, and Ethox HVB.

(16) Phosphates having the following formula:

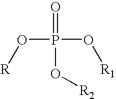

wherein R, R₁, and R₂═H, C₁₋₂₅, or —(CH₂CHR₃O—)ₙH (n=1-200) R₃═H, C₁₋₂, phenyl, and may also be aromatic, unsaturated, saturated, branched, linear, cyclic, alkyl alkoxylate, salt (metal, amine) and R, R₁, and R₂ may be the same or different. R, R₁ and R₂ may be derived from alkoxylated aliphatic alcohols or hydroxylated alkylaromatic. Examples of the above compounds include ERS 00210, Ethfac 102, Ethfac 103, Sodium phosphate monobasic and Ethfac 353.

(17) Tertiary amines having the formula:

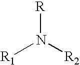

wherein R, R₁, and R₂═H, C₁₋₂₅, or —(CH₂CHR₃O—)ₙH (n=1-200) R₃═H, C₁₋₂, phenyl, and may be aromatic, unsaturated, saturated, branched, linear, or cyclic hydrocarbon groups having up to 25 carbon atoms. R, R₁, and R₂ may be the same or different. Representative compounds include Ethox TAM-2, Ethox CAM-2, dimethyl palmitamine, monoethanol amine, diethanol amine, dimethyl stearamine, 2-ethylhexyl amine, and dimethyl soyamine.

(18) Ammonium quaternaries having the following formula:

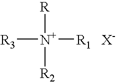

wherein R, R₁, R₂, and R₃═H, C₁₋₂₅, or —(CH₂CHR₃O—)ₙH (n=1-200) R₃═H, C₁₋₂, phenyl, and may be aromatic, unsaturated, saturated, branched, linear or cyclic aromatic groups having up to 25 carbon atoms. R, R₁, R₂, and R₃ may be the same or different, X=halide such a chloride or bromide or iodide or sulfate. Typical examples include stearyldimethylbenzylammonium chloride, triethylmethylammonium chloride, poly-12-hydroxy-stearate-DMAPA-amide DMS quat, Ethox TAM-2 DQ, Ethox SAM-2 DMS quat, Ethox HTAM-15 DQ, Ethox CAM-2 DMS quat, Ethox 3606, Ethox 2878, DMAPA stearamide DMS quat, cocodimethylbenzylammonium chloride, cetylpyridinium chloride, benzyltriethanolammonium chloride, Base 1219, Di-C₁₂₋₁₈-alkyldimethyl quaternary ammonium compounds, chlorides, Soytrimonium chloride, DMAPA lauramide, Ethox DT-15 DMS, tallowpentamethyl-propylenediammonium bismethosulfate, ERS 00212, 1H-Imidazolium, 1-ethyl-2-(8-heptadecenyl)-4,5-dihydro-3-(2-hydroxyethyl)-, ethyl sulfate (salt), and 1-Hexadecanaminium, N,N-dihexadecyl-N-methyl-, chloride.

(19) Copolymers having the following chemical formula:

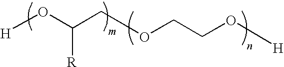

wherein R═H, C₁₋₂, phenyl, m=1-100 and n=1-100 such as Ethox L-61.

(20) Glycerol ester alkoxylates having the formula:

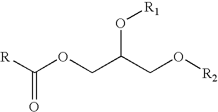

wherein R═H, C₁₋₂₅ alkyl, alkenyl or alkynyl, and may also be aromatic, unsaturated, saturated, branched, linear or cyclic hydrocarbon group having up to 25 carbon atoms, R₁═H, C₁₋₂₅ alkylcarbonyl, or —(CH₂CHR₃O—)ₙR′ (n=1-200), R′ may be C₁₋₂₅ alkylcarbonyl or C₂₋₂₅ alkenyl carbonyl, R₃═H, C₁₋₂, phenyl. When R₁=alkyl carbonyl, the group is C₁₋₂₅ and may include be aromatic, unsaturated, saturated, branched, linear, cyclic hydrocarbon groups having up to 25 carbon atoms. R₂═R or R₁ and may be the same or different. Examples of the above formula include POE(12) glycerol dimyristate, ERS 00206, POE(23) glycerol dimyristate, PEG (23) glyceryl dioleate, and Ethox 2132.

(21) Alcohol alkoxylate esters of the formula:

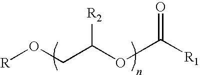

wherein R═H, C₁₋₂₅, and may be aromatic, unsaturated, saturated, branched, linear and cyclic, R₁═H, C₁₋₂₄, and may be aromatic, unsaturated, saturated, branched, linear and cyclic. R₂═H, C₁₋₂, or phenyl. R, R₁ and R₂ may be optionally substituted with halogen or other functional groups selected from the group consisting of hydroxy, carboxy, sulfonate, aldehyde, ketone Examples of compounds corresponding to the above formula include POE(200) I-24 stearate, POE(200) I-24 behenate, and POP(200) I-24 isostearate.

(22) Ester/amide phosphates of the following formula:

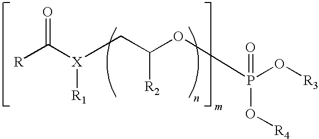

wherein X═N, O and when X=O, R₁ does not exist and when X═N, R₁ is the same as alkoxylate phosphate branch, R═H, C₁₋₂₅ and may be aromatic, unsaturated, saturated, branched, linear or cyclic. R₂═H, C₁₋₂, phenyl, (n=1-200). R₃═H, m, alkyl C₁₋₂₅, alkoxylate and R₄ is same or different than R₃. Typical examples of the above formula includes oleyl MEA phosphate, POP(1.1) laurate phosphate, POP(1.1) laurate phosphate DETA salt, POP(1.1) laurate phosphate MEA salt, POP(1.1) laurate phosphate DEA salt, POP(1.1) laurate and phosphate TEA salt.

Other coupler compounds useful in the practice of the invention includes ERS 00207, glycerol formal, POE(10) glycerol formal, C1214 borate, oleoyl sarcosine, 1,4-cyclohexanedimethanol, lecithin, dendritic polymer, POE(2.2) dimerate, di-TPnB azelate, coco imidazoline, ERS 00211, glycerin, Octyl/decyl-2-ethyl hexanoate, sodium cocoyl hydrolyzed soy protein (and) sorbitol, potassium cocoyl hydrolyzed collagen, and POP(200) I-24 dimerate.

Additional couplers also include betaine surfactants, sultaine surfactants, imidazolines, sulfonates such alkyl, alkenyl and aryl and alkaryl sulfonates. Natural products such as lecithin are also intended to be within the scope of the present invention.

A useful coupling agent is typically an ester such as a glyceryl ester. The term glyceryl ester is intended to include the monoester of glycerin, the diester of glycerin and the triester of glycerin. Also polyglyceryl esters are included within the scope of the instant invention. The esters moieties of the invention are derived from fatty acids, wherein the fatty acid moiety has from about 8 to about 40 carbon atoms, preferably from about 12 to about 22 carbon atoms, more preferably from about 16 to about 20 carbon atoms, most preferably from mixtures containing C₈₋₁₀ and C₁₈ carbon atoms. Non-limiting examples of fatty acids suitable for making the fatty acid gellants include acids such as myristic, palmitic, stearic, oleic, ricinoleic, lauric, arachidic, behenic, linoleic, linolenic, margaric and combinations thereof. These fatty acids are preferably derived from sources such as coconut oil, beef tallow, lanolin, fish oil, beeswax, palm oil, peanut oil, olive oil, cottonseed oil, soybean oil, corn oil, rapeseed oil, rosin acids, greases, and other natural sources, or are derived by synthetic or semisynthetic methods well known to those skilled in the formulation art. The preferred coupling agents are the glyceryl esters derived from C₈₋₁₀ fatty acids, stearic acid and isostearic acid.

The second component of the enhanced gelling agent of the invention is selected from the group consisting of: isocetyl citrate, isoarachidyl citrate, POE(5) diTMP tetra-isostearate, trimethyl soya quaternary ammonium chloride, (C₁₂₋₁₈)dialkyldimethyl-ammonium chloride, soybean oil DEA transester, castor oil, POE(4) cetyl stearyl alcohol, POE(4) decyl alcohol, POE(6) decyl alcohol, POE(2) 2-ethylhexyl alcohol, Epal 1214 phosphate, C₈₋₁₀ triglyceride, CFA/DEA transamide TOFA salt, PPG 2025 TOFA ester, POE(2.5)cocoamine DES quaternary, stearic acid DEA amide, glycerine sesqui-cocoate, TOFA/DEA amide/soap, POE(3.3) CFA/MEA amide, DMSD/TEA DES quaternary, glycerine sesqui-C₈₋₁₀, POE(2) cocoamine, POE(5) castor oil, CFA/DEA transamide, PEG 600 dilaurate, PEG 200 dilaurate, PEG 400 dilaurate, PEG 600 ditallate, PEG 400 ditallate, POE(5) hydrogenated castor oil, POE(15) hydrogenated tallow amine DES quaternary, POE(4) laurate, POE(4) tallate, POE(2) tallow amine, POE(2) tallow amine DES quaternary, sorbitan monostearate (SMS), glycerine dilaurate, glycerine monosoyate, glycerine monostearate (GMS), hydrogenated castor oil (HCO), PEG 200, PEG 400, POE(1.1) laurate, POP(1.1) laurate, sorbitan monolaurate (SML), sorbitan monooleate (SMO), sorbitan monopalmitate (SMP), sorbitan trioleate (STO), soybean oil, triglycerol monostearate, decaglycerol monostearate, and tri-C₁₂₋₁₄ borate ester.

Additional second components may also selected from the group consisting of: 1,4-cyclohexanedimethanol, 1,6-hexane diol, 1-hexadecanaminium, n,n-dihexadecyl-n-methyl-, chloride, 1H-imidazolium, 1-ethyl-2-(8-heptadecenyl)-4,5-dihydro-3-(2-hydroxyethyl)-, ethyl sulfate (salt), 2-ethyl-hexylamine, 2-phenoxyethanol, acylated polypeptides salt, benzyl-triethanolammonium chloride, C₁₂₋₁₈-dialkyldimethylammonium chloride, caprylic/capric 2-ethylhexanoate, caprylic/capric sesquicitrate, caprylic/capric sesquicitrate sodium salt, caprylic/capric sesquiglyceride, caprylic/capric triglyceride, caprylic/capric/isostearyl sesquiglyceride, castor oil, cetylpyridinium chloride, coco imidazoline, coco-amine(2) dimethyl sulfate, coco-amine(2.5) diethyl sulfate, cocodimethylbenzylammonium chloride, coco ethyldimonium etho-sulfate, cyclomethicone+octyl dimethicone ethoxy glucoside, decaglycerol mono-stearate, dendrimer surfactant, diethanol amine, diethanol amine/monoethanol amine octenyl succinate stearate, diethanol amine octenyl succinate stearate, diethanol amine stearate, diethanol amine tallate, diethanolamide isostearate, diethylenetriamine pentaacetic acid, dimethyl cetyl amine, dimethyl coco-amine benzyl chloride, dimethyl soya amine, dimethyl stearyl amine, dimethylaminopropylamine laur-amide, dimethylsoyamine/tri ethanol amine di ethyl sulfate, di-trimethylolpropane C₁₂₋₁₈ tetra ester, ditpnb azelate, dimethylamino-propylamine stearamide dimethyl sulfate, ethylene glycol, ethylene glycol mono-laurate, ethylene glycol monotungate, ethylene oxide propylene oxide copolymer, ethylenediamine tetra-acetic acid, glycerin, glycerol 2-ethylhexanoate, glycerol 2-ethylhexanoate sodium salt, glycerol benzoate, glycerol dilaurate, glycerol dimyristate, glycerol distearate, glycerol formal, glycerol monoisostearate, glycerol monosoyate, glycerol monostearate (gms), glycerol sesqui 2-ethylhexanoate, glycerol sesqui-cocoate, hydrogenated castor oil, isoarachidyl citrate, isocetyl citrate, isolignoceryl(200) behenate, lauric/dea transamide tofa salt, lauryl phosphate, lauryl(3.3) monoethanol amide, monoethanol amine, monoethanol amine octenyl succinate, monoethanol amine octenyl succinate stearate, oleoyl sarcosine, oleyl monoethanolamide phosphate, peg 200, peg 200 dilaurate, peg 400, peg 400 dilaurate, peg 400 ditallate, peg 600 dilaurate, peg 600 ditallate, peg 8000 diisostearate, poe(10) glycerol distearate, poe(10) glycerol formal, poe(12) glycerol dimyristate, poe(15) hydrogenated tallow amine des quat., poe(15) tallow diamine di-methyl sulfate, poe(2) 2-ethyl hexanol, poe(2) bisphenol A, poe(2) cocoamine, poe(2) tallow amine, poe(2) tallow amine diethyl sulfate, poe(2.2) dimerate, poe(200) isolignoceryl alcohol, poe(23) glycerol dimyristate, poe(23) glycerol dioleate, poe(23) lauryl alcohol, poe(25) behenyl alcohol, poe(25) hydrogenated castor oil isostearate, poe(3) lauryl alcohol, poe(3) tridecyl alcohol, poe(3) trimethylolpropane, poe(4) cetyl stearyl alcohol, poe(4) decyl alcohol, poe(4) laurate, poe(4) lauryl alcohol, poe(4) tallate, poe(5) castor oil, poe(5) di-trimethylolpropane tetra-isostearate, poe(5) hydrogenated castor oil, poe(5) hydrogenated castor oil oleate, poe(5) tridecyl phosphate potassium salt, poe(6) decyl alcohol, poe(7) decyl alcohol, poe(70) tristyrenated phenol sulfate, poe(9) isostearate, poly-12-hydroxystearate-dimethylaminopropylamine dimethyl sulfate, pop(2) cetyl alcohol, pop(2.2) dimerate, pop(200) isolignoceryl alcohol, pop(4)poe(1) cetyl alcohol, pop(5) laurate, pop(8) decyl alcohol, potassium dodecenylsuccinate, ppg 2025 tallate, ppg-460, propylene glycol monolaurate, propylene glycol monolaurate phosphate, propylene glycol monolaurate phosphate dea salt, propylene glycol monolaurate phosphate deta salt, propylene glycol monolaurate phosphate mea salt, propylene glycol monolaurate phosphate tea salt, sodium chloride, sodium laureth sulfate, sodium phosphate monobasic, sorbitan monolaurate (sml), sorbitan monooleate (smo), sorbitan monopalmitate (smp), sorbitan monostearate (sms), sorbitan trioleate (sto), soy lecithin, soybean oil, stearylamine(2) dimethyl sulfate, tallowpentamethylpropylenediammonoim bismethosulfate, tridecyl phosphate, triethylmethyl-ammonium chloride, triglycerol monostearate, trilauryl borate, trimethyl soya quaternary ammonium chloride

The solvents of the invention which are gelable by the improved gelling agent of the invention can be polar, non-polar and combinations thereof. The solvents or liquids of the invention may be anhydrous or they may contain water. The solvent preferably comprises one or more anhydrous liquid solvents suitable for the intended application i.e., topical application to human skin, which solvent or combination of liquid solvents are liquid under ambient conditions. The term “anhydrous” as used herein means that the gel composition of the present invention, and the essential or optional components thereof are substantially free of added or free water. From a formulation standpoint, this means that the gel composition of the present invention preferably contain less than about 5%, preferably less than about 3%, more preferably less than about 1%, most preferably zero percent, by weight of free or added water. These liquid anhydrous, solvents may be organic or silicone-containing, volatile or non-volatile, polar or nonpolar, provided that the solvent can form a solution or other homogenous liquid or homogenous liquid dispersion with the selected gellant at the selected gellant concentration at a temperature of from about 28° C. to about 250° C., preferably from about 28° C. to about 100° C., more preferably from about 28° C. to about 78° C. When used in a cosmetic application, the anhydrous liquid solvent preferably has a low viscosity to provide for improved spreading performance on the skin.

The present invention also provides non-anhydrous gels using the same solvents as above but small amounts of water are also permissible. For example some non-anhydrous gels may contain up to 10% water. However, the invention is also applicable to gels where water is a major component or it may be 100% water based.

The preferred solvents are selected from the group consisting of acetone, castor oil, diesel fuel, terpenes, mineral oil, ethanol, synthetic glycerides, ethyl acetate, ethylene glycol, isopropanol, kerosene, methylene chloride, propylene glycol, silicone oil, soybean oil, toluene, water, xylenes, butyl palmitate, isopropyl myristate, isopropyl isostearate, isocetyl isopalmitate. Preferred are nonpolar hydrocarbon solvents which can be cyclic, branched or chain configurations, most preferably branched chain hydrocarbons. A preferred mineral oil is Drakeol 10.

By the expression “mineral oil” is meant a clear colorless nearly odorless and tasteless liquid obtained from the distillation of petroleum. It is also called white oil, white mineral oil, liquid petrolatum, liquid paraffin or white paraffin oil. Mineral oil is a highly refined oily liquid which is commercially available as a technical grade, as a NF (National Formulary) grade and as a USP (United States Pharmacopeia) grade. The specific gravity of mineral oil generally lies between 0.860 and 0.905 gm/cc and its minimum boiling point is 360° C. The number after the commercial name is indicative of the SUS viscosity at 100° F. They are usually free of aromatics and unsaturated compounds.

Other non-limiting examples of hydrocarbon solvents include the isoparaffins available from Exxon Chemical Company, Baytown, Tex. U.S.A, as Isopar M (C₁₃₋₁₄ isoparaffin), Isopar C(C₇₋₈ Isoparaffin), C₈₋₉ Isoparaffin (Isopar E), Isopar G (C₁₀₋₁₁ Isoparaffin), Isopar L (C₁₁₋₁₃ Isoparaffin) and Isopar H(C₁₁₋₁₂ Isoparaffin). Other nonlimiting examples of suitable branched chain hydrocarbons include Permethyl 99A (isododecane), Permethyl 102A (isoeicosane), Permethyl 101A (isohexadecane), and combinations thereof. The Permethyl series are available from Preperse, Inc., South Plainfield, N.J. U.S.A. Other nonlimiting examples of suitable branched chain hydrocarbons include petroleum distillates such as those available from Phillips Chemical as Soltrol 130, Soltrol 170, and those available from Shell as Shell Sol 70, -71, and -2033.

Nonlimiting examples of other suitable nonpolar volatile solvents for use in the anhydrous gel deodorant composition include dibutyl adipate, diisopropyl adipate, dodecane, octane, decane and combinations thereof. Yet another example includes C₁₁₋₁₅ alkanes/cycloalkanes available from Exxon as Exxsol D80.

An additional class of anhydrous liquids usable in making the gels of the invention is the organosilicones. Such organosilicone preferably comprises a modified or organofunctional silicone solvent selected from the group consisting of polyalkylsiloxanes, polyalkarylsiloxanes, polyestersiloxanes, polyethersiloxane copolymers, polyfluorosiloxanes, polyaminosiloxanes, and combinations thereof. These modified silicone solvents must be liquid under ambient conditions, and have a viscosity of less than about 100,000 centistokes, preferably less than about 500 centistokes, more preferably from about 1 centistoke to about 50 centistokes, and even more preferably from about 1 centistoke to about 20 centistokes. These modified solvents are generally known in the chemical arts, some example of which are described in Cosmetics, Science and Technology 27-104 (M. Balsam and E. Sagarin ed. 1972); U.S. Pat. No. 4,202,879, issued to Shelton on May 13, 1980; U.S. Pat. No. 5,069,897, issued to Orr on Dec. 3, 1991; which descriptions are incorporated herein by reference in their entirety.

The modified silicone solvents for use in the compositions of the invention include, but are not limited to, compounds or materials as defined hereinabove and which are generally characterized as follows: silicone polyethers or silicone glycols (such as dimethicone copolyol); silicone alkyl-linked polyethers (such as Goldschmidt EM-90 or EM-97); siloxane surfactants of a pendant/rake/comb configuration, silicone surfactants of a trisiloxane configuration, and silicone surfactants of an ABA/alpha-omega block copolymers (such as polyoxyalkylenes, polyoxyethylene or ethoxylated, polyoxyethylene/polyoxypropylene or ethoxylated/propoxylated); aromatic substituted silicone emollients (such as phenyl, alpha-methyl styryl, styryl, methylphenyl, alkylphenyl); silicone copolymers with other functional groups include: hydrogen, alkyl, methyl, amino, trifluoropropyl, vinyl, alkoxy, aryalkyl, aryl, phenyl, styryl, polyethers, esters, carboxylics; alkylmethyl siloxanes or silicone waxes (such as hexyl, octyl, lauryl, cetyl, stearyl); nonionic functional siloxane copolymers with terminal groups being silanol or trimethylsiloxy; nonionic functional siloxanes with backbone groups being trisiloxane or methicone linked; nonionic silicone surfactants; tetraethoxysilane; tetramethoxysilane; hexamethoxysilicone; methoxytrisiloxane; silicone emulsifiers; silicone or siloxane resins, alkyl silicone resins, polyoxyalkylene silicone resins; MQ Resins such as Shiseido/Shin-etsu, e.g. Japanese Patent Publication JP86143760 or from Walker Chem. 6 MBH (described in EP722970); alkoxysiloxanes; alkoxysilanes; methicones (polymethylalkylsiloxanes); and combinations thereof.

Other nonlimiting examples of suitable modified silicone solvents for use in the compositions of the invention herein include the following modified silicones available from Dow Corning: DC-556 Cosmetic Grade Fluid (phenyl trimethicone); DC-704 Diffusion Pump Fluid (Tetramethyl-Tetraphenyl-Trisiloxane); DC-705 Diffusion Pump Fluid; DC-1784 Emulsion; DC-AF Emulsion; DC-1520-US Emulsion; DC-593 Fluid (Dimethicone [and] Trimethylsiloxysilicate); DC-3225C Fluid (Cyclomethicone [and] Dimethicone Copoly); DC-190 Fluid (Dimethicone Copoly); DC-193 Fluid (Dimethicone Copolyol); DC-1401 (Cyclomethicone [and] Dimethiconol); DC-5200 Fluid (Laurylmethicone Copolyol); DC-6603 Polymer Powder; DC-5640 Powder; DC-Q2-5220 (Dimethicone Copolyol); DC Q2-5324 (Dimethicone Copolyol); DC-250 Cosmetic Wax (Dimethicone Copolyol); DC-2502 Fluid (Cetyl Dimethicone); DC-2503 Wax (Stearyl Dimethicone); DC-1731 Volatile Fluid (Caproyl Trimethicone); DC-580 Wax (Stearoxytrimethylsilane [and] Stearyl Alcohol); DC-1-3563 (Dimethiconal); DC-X2-1286 (Dimethiconol); DC-X2-1146A (Cyclomethicone [and] Dimethiconol); DC-8820 Fluid (Amino functionalized); DC Q5-0158A wax (stearoxytrimethylsilane); DC-Q2-8220 (Trimethylsilyamodimethicone); DC-7224 (Trimethyl-silylamodimethicone); DC-X2-1318 Fluid (Cyclomethicone [and] Vinyldimethicone); DC-QF1-3593A fluid (Trimethylsiloxysilicate) and combinations thereof.

Further nonlimiting examples of suitable modified silicone solvents for use in the compositions of the invention herein include the following modified silicones available from General Electric: GE SF-1023 (Dimethyl-Diphenyl-Siloxane); GE CF-1142 (Methylphenyl Siloxane Fluid); GE SF-1153 (Dimethyl-Diphenyl-Siloxane); GE SF-1265 (Diphenyl-Dimethyl-Siloxane); GE SF-1328; GE SF-1188 (Dimethicone copolyol); GE SF-1188A (Silicone polyether copolymer); GE SF-1288 (silicone polyether copolymer, dimethyl-methyl-3-hydroxypropyl ethoxylated); GE SF-1318 (Methylester Siloxane); GE SF-1328 (silicone surfactant, dimethyl-methyl 3-hydroxypropyl ethoxylated-propoxylated); GE SF-1550 (methylphenyl siloxane, hexamethyl-3-phenyl-3-[[trimethylsily]oxy]trisiloxane); GE SF-1632 (silicone wax); GE SS-4267 (Dimethicone [and] Trimethylsiloxysilicate).

Other nonlimiting examples of suitable modified silicone solvents for use in the compositions of the invention herein include the following modified silicones available from Goldschmidt; Abil EM-90 (silicone emulsifier); Abil EM-97 (polyether siloxane); Abil Wax 9810 (silicone wax or C24-28 methicone); Abil Wax 2434 (Stearoxy Dimethicone); Abil Wax 9800D (Stearyl Dimethicone); and Tegomer H-Si 2111, H-Si 2311, A-Si 2120′, A-Si 2320, C-Si 2141, C-Si 2341, E-Si 2130, E-Si 2330, V-Si 2150, V-Si 2550, H-Si 6420, H-Si 6440, H-Si 6460 (Alpha-Omega Dimethicone Copolymers).

Other nonlimiting examples of suitable modified silicone solvents for use in the pharmaceutical compositions herein include the following: Masil 756 from PPG Industries (Tetrabutoxypropyl Trisiloxane); bis-phenylhexamethicone (available as Silbione Oils 70633 V30 from Rhone-Poulenc); Sibione Oils 70646 (dimethicone copolyols from Rhone-Poulenc); Silicone L-711, L-720, L-721 and L-722 (dimethicone copolyols from Union Carbide); Silicone L-7000, L-7001, L-7002, L-7004, L-7500, L-7600, L-7602, L-7604, L-7605, and L-7610 (dimethicone copolyols from Union Carbide); Unisil SF-R (dimethiconol from UPI); Silicate Cluster from Olin (Tris[tributoxysiloxy]methylsilane); silicone copolymer F-754 (dimethicone copoly from SWS Silicones); and combinations thereof.

The anhydrous and non-anhydrous gel compositions of the present invention may also contain one or more polar solvents. Numerous combinations of solvents may be used in the practice of the invention. The gels may contain only non-polar solvents, however it may also be made of polar solvents only or combinations of non-polar and polar. The concentration of the polar solvent in the anhydrous gel composition will vary with the specific combination of polar solvent, gellant, and optional other solvents in the composition. The amounts of each solvent are selected such that the desired gels are obtained depending on the properties desired by the formulator. The present invention provides numerous examples in the tables so it would be easy to appreciate that the selection of substrate or combinations of substrates, metallic soap, coupling agent and other ingredients is easily made to achieve the desired gel consistencies.

Nonlimiting examples of suitable polar solvents for use in the anhydrous and non-anhydrous gel compositions include water, monohydric alcohols, polyhydric alcohols, and combinations thereof, examples of which include C₁ to C₂₀ monohydric alcohols, preferably C₂ to C₈ monohydric alcohols, and polypropylene glycols and polyethylene glycols having from 2 to 7 repeating ethoxylate or propoxylate groups, and polyglycerols having from 2 to 16 repeating glycerol moieties.

Specific examples of polar solvents suitable for use in the anhydrous and non-anhydrous gel composition include, but are not limited to, glycerin, propylene glycol, dipropylene glycol, ethanol, water, tripropylene glycol, butylene glycol, propylene glycol methyl ether, dipropylene glycol methyl ether, and combinations thereof. Most preferred is glycerin.

As used herein, the term “gel” is defined when a substrate viscosity has been increased to provide viscous liquids, a solid or semi-solid colloid containing considerable quantities of the liquid which is being gelled by the gellant compositions of the invention. The particles in the gel are linked in a coherent mesh work which immobilizes the liquid. The gels within the scope of the present invention may be described as gels that have a firm jelly-like consistency; that is, by tapping the gel lightly, it will vibrate and return to its original configuration. The instant invention also defines a “gel” as any viscosity increase afforded a substrate up to and including a hard solid.

The resulting gelled products of the invention typically have viscosities in the range of 1 cps to 10 million cps, albeit viscosities in the ranges of 0.5 million cps to 1 million cps and viscosities of greater than 10 million cps can be achieved.

Concentrations of the (substrate) solvent in the gel will vary with the type of solvent selected, the type of gelling agent used in combination with the solvent, and the solubility of the selected gelling agent in the selected solvent, and so forth. Preferred concentrations of the solvent ranges from about 5% to about 99.9%, preferably from about 30% to about 95%, more preferably from about 80% to about 90%, by weight of the composition. The usage level is driven by the desire to gel the most substrate using the minimal level of gel system.

The compositions of the present invention may be made by any of the methods known in the art for formulating gel compositions. As will be apparent to those skilled in the art, the particular method will be dependent upon the selection of the specific types and amounts of the components employed.

In general, the compositions of the present invention can be prepared by mixing the polar solvent or nonpolar solvent, or a combination of polar and non-polar solvents and any other optional solvents. Add gellant with agitation and heat the mixture to a temperature of from about 75° C. to about 100° C. to allow the gellant to melt and form a substantially clear or translucent liquid. The resulting solution may be poured into an appropriate container or dispenser at about 70° C. and allowed to solidify within the container or dispenser by cooling or allowing to cool the contained composition to ambient temperature. Of course the pour point may be higher than 70° C. depending on the substrates and gellants selected.

The gel product/compositions of the present invention may be characterized in terms of product hardness, and/or a rheology profile defined by a ratio of elastic to viscous moduli. Each of these characteristics is defined in accordance with the methodologies and other limitations described hereinafter.

The gel compositions of the present invention preferably have a product hardness of from about 500 gram force to about 5000 gram force, more preferably from about 750 gram force to about 2,000 gram force, and most preferably from about 800 gram force to about 1400 gram force. The term “product hardness” as used herein is a reflection of how much force is required to move a penetration cone a specified distance and at a controlled rate into a gel-solid stick composition under the following test conditions. Higher values represent harder product, and lower values represent softer product. These values are measured at 27° C., 15% relative humidity, using a TA-XT2 Texture Analyzer, available from Texture Technology Corp., Scarsdale, N.Y., U.S.A. The product hardness value as used herein represents the amount of force required to move a standard 45 angle penetration cone through the composition for a distance of 10 mm at a rate of 2 mm/second. The standard cone is available from Texture Technology Corp., as part number TA-15, and has a total cone length of about 24.7 mm, angled cone length of about 18.3 mm, a maximum diameter of the angled surface of the cone of about 15.5 mm. The cone is a smooth, stainless steel construction and weighs about 17.8 grams.

The gel compositions of the present invention are preferably gel-solids having the select rheology profile defined herein. This rheology is defined herein in terms of the elastic (G′) to viscous (G″) moduli ratio (G′/G″) of the gel-solid stick composition. To provide the desired rheology, the gel compositions preferably have a G′/G″ ratio of from about 1 to about 100, more preferably from about 1 to about 70, most preferably from about 1 to about 20, even more preferably from about 1 to about 10. This ratio represents the extent to which the gel compositions herein are preferred to exhibit solid character and the extent to which the compositions are preferred exhibit liquid or fluid character, and specifically refers to the numerical ratio G′/G″ as determined by the following methodology.

In particular, the elastic modulus is a measurement which correlates with the solid character of the gel compositions herein, and the viscous modulus is a measurement which correlates with the fluid or liquid character of the gel compositions herein. Measurements for G′ and G″ for purpose of defining the composition of the present invention are determined under ambient conditions using conventional techniques well known in the formulation arts. For example, a Bohlin Stress-Strain Rheometer, available from Bohlin Reologi, Cranberry, N.J., can be used using a cone (about 1) and plate configuration. About 1.0 mg of the product is carefully removed for the composition with minimal application of shear force and is then placed between the cone and plate fixtures for measurement of G′ and G″.

Furthermore, the gel compositions of the invention can advantageously be clear, transparent or translucent. The terms “translucent” and “transparent” can be understood by the conventional definitions given in the dictionary. Thus, a translucent composition allows light to pass through without, however, allowing the contours of objects to be sharply distinguished. A transparent composition allows light to pass through easily and allows objects to be sharply distinguished through its thickness.

The gel compositions of the present invention may also be formulated into cosmetic compositions and optionally include cosmetic actives such as moisturizers and/or skin protectants. Suitable cosmetic compositions include lipsticks gels, bar soaps, lip balms, soft gels, creams, makeup, lotions, roll-ons, facial moisturizers, or gel sticks and the like. Useful moisturizers and/or skin protectants are disclosed in the Federal Register Vol. 48, No 32 and include aloe vera, allantoin, aluminum hydroxide gel, bismuth subnitrate, boric acid, calamine, cocoa butter, corn starch, dimethicone, glycerin, lanolin, kaolin, live yeast cell derivative, petrolatum, shark liver oil, sodium bicarbonate, sulfur, tannic acid, white petrolatum, zinc acetate, zinc carbonate and zinc oxide and mixtures thereof. The skin protectant and/or moisturizer preferably comprise from about 0.001% to about 2%, more preferably from about 0.01% to about 1% of the gel composition.

The novel gellant system of the invention can also be used to gel water.

The following examples further describe and demonstrate embodiments within the scope of the present invention. These examples are solely for the purpose of illustration and are not to be construed as limitations of the present invention as many variations are possible without departing from the spirit or scope thereof. All levels and ratios are by weight of the total composition, unless otherwise indicated. By the phrase “ambient temperature” as used herein, refers to surrounding conditions under about one atmosphere of pressure, at about 50% relative humidity, and at about 25° C., unless otherwise specified.

### Example 1

To a beaker containing 50 g of Drakeol 10, add 2 g sodium stearate. Heat contents to a temperature sufficient to afford incorporation of the metallic soap into the substrate. Upon cooling to ambient temperature, some gelatinous material is observed, but liquid (syneresis) is still found between the gel portion and the vessel walls.

### Example 2

To a beaker containing 50 g Drakeol 10, add 10 g Ethox 4075. Heat contents to a temperature sufficient to afford incorporation of coupling agent into the substrate. Upon cooling, a one-phase liquid is observed.

### Example 3

This is a 50/50 mix of Example I and Example 2. Combine the contents of the beakers and heat to a temperature sufficient to afford a liquid. This is important, as the product is a gelatinous material. If the temperature is too low, the gel thus formed hinders complete mixing. Heating the beakers separate prior to combining helps with the mixing. Upon cooling a firm, translucent gel is observed.

### Example 3A

### Preparation of Gels Using Anionic Surfactants

Each example was prepared in a manner analogous to that for example 3 above. All ingredients were combined in a single vessel with mixing and then heated until clear and held at that temperature with mixing for approximately 5-10 minutes and then cooled to RT. In each example Ethox 4075 was added last. The viscosity of each mixture was measured before addition of Ethox and after recooling to RT. Viscosity measurements were made using a Brookfield DV-II+Digital RVT Viscometer using spindle TE @ 5 rpm. The mixtures may increase in viscosity with the addition of Ethox 4075 at RT before heating but this increase appears to be enhanced by heating and recooling to RT. See further Examples 582-585 in Table 1 below.

### Example 4

This is the one-step gelation of Example 3. In a beaker, add 50 g Drakeol 10 and heat. In a second beaker, add 10 g Ethox 4075 and 2 g sodium stearate. Heat this beaker to a temperature sufficient to afford mixing. Once a one-phase system is obtained, add the entire contents of the beaker to the heated beaker containing the Drakeol 10. If some gelation occurs, thus hindering mixing, keep heating the mass until the gel brakes. Upon cooling a firm, translucent gel is observed.

### Example 5

To a beaker containing 50 g soybean oil, add 10 g glyceryl monosoyate. Heat to a temperature sufficient to afford mixing. Upon cooling, a one-phase liquid is observed.

### Example 6

To a beaker containing 50 g soybean oil, add 2 g sodium stearate. Heat to a temperature sufficient to afford mixing. Upon cooling, a one-phase liquid is observed.

### Example 7

In a beaker add 50 g soybean oil and heat. In a separate beaker add 10 g glyceryl monosoyate and 2 g sodium stearate. Heat to a temperature sufficient to afford mixing. Add the entire contents of this beaker into the beaker containing the hot soybean oil. If gelation hinders mixing, keep heating the material until mixing can be accomplished. Upon cooling a translucent gel is observed.

The following additional Examples are summarized in Table 1.

TABLE 1

| Exam- | | | | | |
|---|---|---|---|---|---|
| ple No. | Substrate | Coupling agent | Gelling agent | Ratio (grams) | Notes |
| 8 | Drakeol 10* | Ethox 4075 | | 50:5 | Did not gel |
| 9 | Drakeol 10 | Ethox 4075 | Sodium carbonate | 50:5:1 | Sodium carbonate |
| | | | | | insoluble in system |
| 10 | Drakeol 10 | Ethox 4075 | Sodium carbonate | 50:5:2 | Sodium carbonate |
| | | | | | insoluble in system |
| 11 | Drakeol 10 | Ethox 4075 | Sodium carbonate | 50:5:3 | Sodium carbonate |
| | | | | | insoluble in system |
| 12 | Drakeol 10 | Ethox 4075 | Sodium carbonate | 50:5:4 | Sodium carbonate |
| | | | | | insoluble in system |
| 13 | Drakeol 10 | Ethox 4075 | Aluminum | 50:5:1 | Did not gel |
| | | | isopropoxide | | |
| 14 | Drakeol 10 | Ethox 4075 | Sodium stearate | 50:5:0.5 | Clear stiff gel |
| 15 | Drakeol 10 | Ethox 4075 | Sodium stearate | 50:10:1 | Hard gel |
| 16 | Drakeol 10 | Ethox 4075 | Sodium stearate | 50:1:0.1 | Soft gel |
| 17 | Drakeol 10 | Ethox 4075 | Sodium oxalate | 50:5:0.12 | Sodium oxalate insoluble |
| | | | | | in system |
| 18 | Drakeol 10 | Ethox 4075 | Sodium oxalate | 50:5:0.23 | Sodium oxalate insoluble |
| | | | | | in system |
| 19 | Drakeol 10 | Ethox 4075 | Sodium dodecyl sulfate | 50:5:0.5 | Soft opaque gel |
| 20 | Drakeol 10 | Ethox 4075 | Sodium EDTA | 50:5:6 | Sodium EDTA insoluble |
| | | | | | in system |
| 21 | Drakeol 10 | Ethox 4075 | Sodium EDTA | 50:5:1.5 | Sodium EDTA insoluble |
| | | | | | in system |
| 22 | Drakeol 10 | Ethox 4075 | Borax | 50:5:0.33 | Borax insoluble in |
| | | | | | system |
| 23 | Drakeol 10 | Ethox 4075 | Calcium acetate | 50:5:0.27 | Calcium acetate |
| | | | | | insoluble in system |
| 24 | Drakeol 10 | Ethox 4075 | Potassium acetate | 50:5:0.17 | Did not gel |
| 25 | Drakeol 10 | Ethox 4075 | Dipotassium dodecenyl | 50:5:0.62 | Did not gel |
| | | | succinate | | |
| 26 | Drakeol 10 | Ethox 4075 | Dipotassium dodecenyl | 50:5:0.31 | Did not gel |
| | | | succinate | | |
| 27 | Drakeol 10 | Ethox 4075 | Sodium azelate | 50:5:0.4 | Sodium azelate insoluble |
| | | | | | in system |
| 28 | Drakeol 10 | Ethox 4075 | Sodium azelate | 50:5:0.2 | Sodium azelate insoluble |
| | | | | | in system |
| 29 | Drakeol 10 | Ethox 4075 | Disodium dimerate | 50:5:1.15 | Did not gel |
| 30 | Drakeol 10 | Ethox 4075 | Sodium stearate | 50:5:1 | Hard opaque gel |
| 31 | Drakeol 10 | Ethox 4075 | Sodium stearate | 50:5:0.5 | Clear |
| 32 | Drakeol 10 | Ethox 4075 | Sodium stearate | 50:5:1 | Translucent |
| 33 | Drakeol 10 | Ethox 4075 | Sodium stearate | 50:5:1.5 | Translucent |
| 34 | Drakeol 10 | Ethox 4075 | Sodium stearate | 50:10:0.5 | Did not gel |
| 35 | Drakeol 10 | Ethox 4075 | Sodium stearate | 50:10:1 | Clear |
| 36 | Drakeol 10 | Ethox 4075 | Sodium stearate | 50:10:2 | Translucent |
| 37 | Drakeol 10 | Ethox 4075 | Sodium stearate | 50:10:4 | Hard opaque gel |
| 38 | Drakeol 10 | Ethox 4075 | Sodium stearate | 50:3:0.3 | Soft clear gel |
| 39 | Drakeol 10 | Ethox 4075 | Sodium stearate | 50:3:0.6 | Translucent |
| 40 | Drakeol 10 | Ethox 4075 | Sodium stearate | 50:3:1 | Opaque |
| 41 | Drakeol 10 | | Sodium stearate | 50:2 | Loose gel |
| 42 | Drakeol 10 | Ethox 4075 | | 50:10 | Did not gel |
| 43 | Drakeol 10 | Ethox 4075 | Sodium | 50:5:1:5 | Two layers |
| | | | stearate/Ethylene | | |
| | | | carbonate | | |
| 44 | Drakeol 10 | Ethox 4075 | Sodium stearate/ | 50:5:1:5 | Two layers |
| | | | Propylene carbonate | | |
| 45 | Drakeol 10 | Blend of examples 41 & | | | Gel |
| | | 42 | | | |
| 46 | Drakeol 10 | Ethox 4075 | Sodium stearate | 50:10:1 | Clear gel |
| 47 | Drakeol 10 | Ethox 4075 | Sodium stearate | 50:5:1 | Translucent |
| 48 | | Ethox 4075 | Sodium stearate | 100:10 | Thick (solid) gel turns |
| | | | | | black |
| 49 | | Ethox 4075 | Stearic acid/sodium | 100:9.3:1.35 | Soft gel no discoloration |
| | | | hydroxide/water | | |
| 50 | Drakeol 10 | Example 49 | | 50:11 | Did not gel |
| 51 | Drakeol 10 | Ethox 4075 | Borax | 50:10:1 | Did not gel |
| 52 | Water | Ethox 4075 | Sodium stearate | 10:50:5 | Paste |
| 53 | Drakeol 10 | Ethox 4073 | Sodium stearate | 50:10:1 | Slight thickening |
| 54 | Drakeol 10 | Ethox 4073 | Sodium stearate | 50:10:2 | Soft gel |
| 55 | Drakeol 10 | Ethox 4073 int. | Sodium stearate | 50:10:2 | Two layers |
| 56 | Drakeol 10 | Ethox 4076 | Sodium stearate | 50:10:2 | Hard translucent gel |
| 57 | Drakeol 10 | Ethox 4075 | Sodium stearate | 50:10:2 | Same as control |
| 58 | Drakeol 10 | Ethox 4075 | Sodium | 50:6:0.6:2 drops | Clear hard gel |
| | | | stearate/glycolic acid | | |
| 59 | Drakeol 10 | Ethox 4075 | Sodium stearate | 100:10:2 | Repeat of example 32 |
| 60 | Drakeol 10 | Ethox 4075 | Sodium stearate | 100:20:2 | Repeat of example 35 |
| 61 | Drakeol 10 | Ethox 4075 | Sodium stearate | 100:20:8 | Repeat of example 37 |
| 62 | Drakeol 10 | Ethox 4075 | Sodium stearate | 100:6:0.6 | Repeat of example 38 |
| 63 | α-Pinene | Glycerol sesqui-2-ethyl | Sodium stearate | 8:1:0.25:0.25:0.25 | 2-phase |
| | | hexanoate/glycerine/ | | | |
| | | ethylene glycol | | | |
| 64 | Drakeol 10 | Ethox 4075 | Stearic acid/sodium | 50:10:2:0.14 | Did not gel. |
| | | | hydroxide/water | | |
| 65 | Drakeol 10 | Sorbitan monooleate | Sodium stearate | 50:10:2 | Translucent solid gel |
| 66 | Drakeol 10 | Sorbitan monolaurate | Sodium stearate | 50:10:2 | Translucent solid gel |
| 67 | Drakeol 10 | Glycerine monostearate | Sodium stearate | 50:10:2 | Soft solid opaque |
| 68 | Drakeol 10 | Glycerine monosoyate | Sodium stearate | 50:10:2 | Translucent solid gel |
| 69 | Drakeol 10 | Sorbitan monopalmitate | Sodium stearate | 50:10:2 | Opaque soft solid |
| 70 | Drakeol 10 | Glycerine dilaurate | Sodium stearate | 50:10:2 | Opaque gel |
| 71 | Drakeol 10 | PEG 200 | Sodium stearate | 50:10:2 | Two layers does produce |
| | | | | | soft gel |
| 72 | Drakeol 10 | PEG 400 | Sodium stearate | 50:10:2 | Did not gel |
| 73 | Drakeol 10 | Ethox 2156 | Sodium stearate | 50:10:2 | Did not gel |
| 74 | Drakeol 10 | Ethox DL-5 | Sodium stearate | 50:10:2 | Loose gel visc. 2200 cPs |
| 75 | Drakeol 10 | Ethox DL-9 | Sodium stearate | 50:10:2 | Loose gel visc. 4750 cPs |
| 76 | Drakeol 10 | Ethox DL-14 | Sodium stearate | 50:10:2 | Loose gel visc. 4500 cPs |
| 77 | Drakeol 10 | Ethox DO-9 | Sodium stearate | 50:10:2 | Loose gel |
| 78 | Drakeol 10 | Ethox DO-14 | Sodium stearate | 50:10:2 | Did not gel |
| 79 | Drakeol 10 | ERS 00204 | Sodium stearate | 50:10:2 | Loose gel |
| 80 | Drakeol 10 | ERS 00205 | Sodium stearate | 50:10:2 | Did not gel |
| 81 | Drakeol 10 | Ethsorb S | Sodium stearate | 50:10:2 | Hard opaque gel |
| 82 | Drakeol 10 | Sorbitan trioleate | Sodium stearate | 50:10:2 | Soft gel |
| 83 | Drakeol 10 | Ethox 4075 | Sodium stearate | 50:10:2 | Hard opaque gel |
| 84 | Drakeol 10 | Ethox 2610 | Sodium stearate | 50:10:2 | Did not gel |
| 85 | Drakeol 10 | Ethox 4074 | Sodium stearate | 50:10:2 | Firm translucent gel |
| 86 | Drakeol 10 | Ethox 4074 | Sodium stearate | 50:10:1 | Medium translucent gel |
| 87 | Drakeol 10 | Soybean oil | Sodium stearate | 50:10:2 | Did not gel |
| 88 | Drakeol 10 | Hydrogenated castor oil | Sodium stearate | 50:10:2 | Soft gel |
| 89 | Drakeol 10 | Ethox CO-5 | Sodium stearate | 50:10:2 | Medium opaque gel |
| 90 | Drakeol 10 | Ethox HCO-5 | Sodium stearate | 50:10:2 | Medium opaque gel |
| 91 | Drakeol 10 | Castor oil | Sodium stearate | 50:10:2 | Did not gel |
| 92 | Soybean oil | Ethox 4074 | Sodium stearate | 50:10:2 | Medium translucent gel |
| 93 | Soybean oil | Glycerine monosoyate | Sodium stearate | 50:10:2 | Medium translucent gel |
| 94 | Soybean oil | Glycerine monosoyate | Sodium stearate | 50:10:2 | Hard opaque gel |
| 95 | Soybean oil | Glycerine monosoyate | | 50:10 | Did not gel |
| 96 | Soybean oil | | Sodium stearate | 50:2 | Did not gel |
| 97 | Soybean oil | Ethox 4074 | Sodium stearate | 50:20:2 | Hard translucent gel |
| 98 | Castor oil | Ethox 4074 | Sodium stearate | 50:10:2 | Medium opaque gel |
| 99 | Castor oil | Ethox 4074 | Sodium stearate | 50:20:2 | Medium opaque gel |
| 100 | Drakeol 10 | POE(1.1) laurate | Sodium stearate | 50:10:2 | Soft opaque gel |
| 101 | Ethox 2156 | Ethox 4075 | Sodium stearate | 50:10:2 | Soft opaque gel |
| 102 | Ethox 2156 | Ethox 4075 | Sodium stearate | 50:20:2 | Medium opaque gel |
| 103 | Ethox 2156 | Ethox 4075 | Sodium stearate | 50:20:4 | Soft opaque gel |
| 104 | Ethox 2156 | Ethox 4074 | Sodium stearate | 50:10:2 | Soft opaque gel |
| 105 | Ethox 2156 | Ethox 4074 | Sodium stearate | 50:20:2 | Medium opaque gel |
| 106 | d-Limonene | Ethox 4075 | Sodium stearate | 5:1:0.1 | Loose gel |
| 107 | d-Limonene | Ethox 4075 | Sodium stearate | 5:2:0.2 | Soft translucent gel |
| 108 | d-Limonene | Ethox 4075 | Sodium stearate | 5:4.5:0.5 | Hard translucent gel |
| 109 | d-Limonene | Ethox 4074 | Sodium stearate | 5:2:0.2 | Did not gel |
| 110 | d-Limonene | Ethal DA-6 | Sodium stearate | 5:2:0.2 | Soft translucent gel |
| 111 | d-Limonene | Ethal DA-4 | Sodium stearate | 5:2:0.2 | Soft translucent gel |
| 112 | d-Limonene | Ethal EH-2 | Sodium stearate | 5:2:0.2 | Soft translucent gel |
| 113 | d-Limonene | | Sodium stearate | 5:0.2 | Medium clear gel |
| 114 | d-Limonene | | Sodium stearate | 5:0.4 | Hard clear gel |
| 115 | Diesel fuel | Ethox 4074 | Sodium stearate | 50:10:2 | Medium translucent gel |
| 116 | Kerosene | Ethox 4074 | Sodium stearate | 50:10:2 | Hard clear gel |
| 117 | Drakeol 10 | Ethox 3051 | Sodium stearate | 50:10:2 | Medium opaque gel |
| 118 | Drakeol 10 | Ethox 4075 | Sodium 12- | 50:10:2 | Hard opaque gel |
| | | | hydroxystearate | | |
| 119 | Drakeol 10 | Ethox 4075 | Sodium 12- | 50:20:2 | Hard translucent gel |
| | | | hydroxystearate | | |
| 120 | Drakeol 10 | Ethox 4075 | Sodium oleate | 50:10:2 | Firm translucent gel - |
| | | | | | unique feel |
| 121 | POE(1.1) laurate | | Potassium hydroxide | | Hard soap |
| 122 | POE(1.1) laurate | | Sodium hydroxide | | Hard soap |
| 123 | Drakeol 10 | Example 121 | | 50:10 | Medium opaque gel |
| 124 | Drakeol 10 | Example 122 | | 50:11 | Hard opaque gel |
| 125 | Drakeol 10 | POP(1.1) laurate | Sodium stearate | 50:10:2 | Thickened - did not gel |
| 126 | Drakeol 10 | Ethox 4075 | Aluminum | 50:10:2 | Did not gel |
| | | | isopropoxide | | |
| 127 | Drakeol 10 | Ethox 4075 | Potassium stearate | 50:10:2 | Did not gel |
| 128 | d-Limonene | | Sodium 2- | 5:0.2 | Did not gel |
| | | | ethylhexanoate | | |
| 129 | Drakeol 10 | POE(1.1) laurate | Sodium stearate | 50:10:2 | Soft opaque gel |
| 130 | Drakeol 10 | POE(1.1) laurate | Sodium stearate | 50:20:2 | Hard opaque gel |
| 131 | Drakeol 10 | POP(1.1) laurate | Sodium stearate | 50:10:2 | Medium opaque gel |
| 132 | Drakeol 10 | POP(1.1) laurate | Sodium stearate | 50:20:2 | Hard opaque gel |
| 133 | d-Limonene | | Sodium stearate | 50:4 | Hard translucent gel |
| 134 | d-Limonene | | Sodium stearate | 100:8 | Hard translucent gel |
| 135 | Ethox 2156 | Ethox 4074 | Sodium stearate | 100:40:4 | Opaque gel |
| 136 | Silicone oil 20 cSt | Ethox 4074 | Sodium stearate | 50:10:2 | Two-phase, did not gel |
| 137 | Dow Corning | Ethox 4074 | Sodium stearate | 50:10:2 | Did not gel |
| | 1107 fluid** | | | | |
| 138 | Drakeol 10 | Amide PF | Sodium stearate | 50:10:2 | Hard opaque gel |
| 139 | Drakeol 10 | Ethox 2449 | Sodium stearate | 50:10:2 | Two-phase, soft gel |
| 140 | Drakeol 10 | Ethox COA | Sodium stearate | 50:10:2 | Two-phase |
| 141 | Drakeol 10 | Ethox CAM-2 | Sodium stearate | 50:10:2 | Hard clear gel |
| 142 | Drakeol 10 | ERS 00209 | Sodium stearate | 50:10:2 | Two-phase, loose gel |
| 143 | Drakeol 10 | Ethox 3166 | Sodium stearate | 50:10:2 | Hard opaque gel |
| 144 | Drakeol 10 | Ethox 2984 | Sodium stearate | 50:10:2 | Hard opaque gel |
| 145 | Drakeol 10 | Ethox 2878 | Sodium stearate | 50:10:2 | Two-phase, soft clear gel |
| 146 | Drakeol 10 | Ethox 3606 | Sodium stearate | 50:10:2 | Loose gel (3606 is only |
| | | | | | 35% active) |
| 147 | Drakeol 10 | Ethox TAM-2 | Sodium stearate | 50:10:2 | Hard clear/translucent |
| | | | | | gel |
| 148 | Drakeol 10 | Ethox TAM-2 DQ | Sodium stearate | 50:10:2 | Hard clear gel |
| 149 | Drakeol 10 | Ethox HTAM-15 DQ | Sodium stearate | 50:10:2 | Two-phase, medium |
| | | | | | opaque gel |
| 150 | Drakeol 10 | Ethox TAM-2 | Stearic acid | 50:1.5:2 | Opaque fluid |
| 151 | Drakeol 10 | Ethox TAM-2 DQ | | 50:10 | Two-phase, did not gel |
| 152 | Drakeol 10 | ERS 00210 | Sodium stearate | 50:10:2 | Loose opaque gel |
| 153 | Drakeol 10 | ERS 00210 | Sodium stearate | 50:10:2 | Medium gel (repeat of |
| | | | | | example 153) |
| 154 | α-Pinene | Glycerol sesqui-2- | Sodium stearate | 8:1.5:0.5 | Soft translucent gel |
| | | ethylhexanoate | | | |
| 155 | Drakeol 10 | Ethfac 102 | Sodium stearate | 50:10:2 | Hard opaque gel |
| 156 | Ethanol | Ethox 4074 | Sodium stearate | 50:10:2 | Hard opaque gel |
| 157 | Ethanol | Ethox CAM-2 DQ | Sodium stearate | 50:10:2 | Hard translucent gel |
| 158 | Drakeol 10 | Di-C12-18-alkyl | Sodium stearate | 50:10:2 | Hard translucent gel |
| | | dimethyl quaternary | | | |
| | | ammonium compounds, | | | |
| | | chlorides | | | |
| 159 | Drakeol 10 | Soytrimonium chloride | Sodium stearate | 50:10:2 | Two-phase - top layer |
| | | | | | is a soft gel |
| 160 | Drakeol 10 | Ethox ML-5 | Sodium stearate | 50:10:2 | Medium opaque gel |
| 161 | Drakeol 10 | Ethox MO-5 | Sodium stearate | 50:10:2 | Medium opaque gel |
| 162 | Drakeol 10 | Ethal 368 | Sodium stearate | 50:10:2 | Medium opaque gel |
| 163 | Drakeol 10 | ERS 00207 | Sodium stearate | 50:10:2 | Did not gel |
| 164 | Isopropanol | Ethox 4074 | Sodium stearate | 50:10:2 | Some gel, need to work |
| | | | | | on mixing |
| 165 | Acetone | Ethox 4074 | Sodium stearate | 50:10:2 | Some gel, need to work |
| | | | | | on mixing |
| 166 | Toluene | Ethox 4074 | Sodium stearate | 50:10:2 | Soft translucent gel |
| 167 | Methylene | Ethox 4074 | Sodium stearate | 50:10:2 | Some gel, need to work |
| | chloride | | | | on mixing |
| 168 | Ethyl acetate | Ethox CAM-2 DQ | Sodium stearate | 50:10:2 | Soft opaque gel |
| 169 | Ethylene glycol | Ethox CAM-2 DQ | Sodium stearate | 50:10:2 | Thick opaque liquid |
| 170 | Isopropanol | Ethox 4074 | Sodium stearate | 50:10:2 | No good |
| 171 | Isopropanol | Ethox 4074 | Sodium stearate | 50:10:2 | Add 10 g water, hard |
| | | | | | opaque gel |
| 172 | Isopropanol | | Sodium stearate | 50:2 | Add 10 g water, hard |
| | | | | | translucent gel |
| 173 | Isopropanol | | Stearic acid/sodium | 50:1.85:0.31 | Add 10 g water, hard |
| | | | hydroxide | | opaque gel |
| 174 | Drakeol 10 | | Sodium stearate | 50:2 | Unstable clear gel |
| 175 | Drakeol 10 | Ethox 4074 | Aluminum stearate 404 | 50:10:2 | Clear liquid |
| 176 | Drakeol 10 | Ethox 4074 | Aluminum stearate NF | 50:10:2 | Translucent liquid |
| 177 | Drakeol 10 | Ethox 4074 | Calcium stearate | 50:10:2 | Translucent liquid |
| 178 | Drakeol 10 | Ethox 4074 | Lithium stearate | 50:10:2 | Opaque liquid |
| 179 | Drakeol 10 | Ethox 4074 | Magnesium stearate | 50:10:2 | Opaque liquid |
| 180 | Drakeol 10 | Ethox 4074 | Zinc stearate | 50:10:2 | Opaque liquid |
| 181 | Drakeol 10 | Triethylmethylammonium | Sodium stearate | 50:10:2 | Soft translucent gel |
| | | chloride | | | |
| 182 | Drakeol 10 | Poly-12-hydroxy- | Sodium stearate | 50:10:2 | Medium translucent gel |
| | | stearate-DMAPA amide | | | |
| | | DMS quat. | | | |
| 183 | Drakeol 10 | Cocodimethylbenzylammonium | Sodium stearate | 50:10:2 | Two-phase, did not gel |
| | | chloride | | | |
| 184 | Drakeol 10 | Benzyltriethanolammonium | Sodium stearate | 50:10:2 | Two-phase, did not gel |
| | | chloride | | | |
| 185 | Drakeol 10 | Cetylpyridinium chloride | Sodium stearate | 50:10:2 | Medium opaque gel |
| 186 | Drakeol 10 | DMAPA stearamide | Sodium stearate | 50:10:2 | Two-phase, soft gel on |
| | | DMS quat. | | | top |
| 187 | Drakeol 10 | Stearyldimethylbenzylammoinium | Sodium stearate | 50:10:2 | Two-phase, did not gel |
| | | chloride | | | |
| 188 | Drakeol 10 | Ethox CAM-2 DMS | Sodium stearate | 50:10:2 | Two-phase soft gel |
| | | quat. 85% in propylene | | | |
| | | glycol | | | |
| 189 | Drakeol 10 | Ethox CAM-2 | Aluminum stearate 404 | 50:10:2 | Clear liquid |
| 190 | Drakeol 10 | Ethox CAM-2 | Aluminum stearate NF | 50:10:2 | Translucent liquid |
| 191 | Drakeol 10 | Ethox CAM-2 | Calcium stearate | 50:10:2 | Translucent liquid |
| 192 | Drakeol 10 | Ethox CAM-2 | Lithium stearate | 50:10:2 | Opaque liquid |
| 193 | Drakeol 10 | Ethox CAM-2 | Magnesium stearate | 50:10:2 | Clear liquid |
| 194 | Drakeol 10 | Ethox CAM-2 | Zinc stearate | 50:10:2 | Translucent liquid |
| 195 | Drakeol 10 | Ethox TAM-2 DQ | Aluminum stearate 404 | 50:10:2 | Two-phase, did not gel |
| 196 | Drakeol 10 | Ethox TAM-2 DQ | Aluminum stearate NF | 50:10:2 | Two-phase, did not gel |
| 197 | Drakeol 10 | Ethox TAM-2 DQ | Calcium stearate | 50:10:2 | Two-phase, did not gel |
| 198 | Drakeol 10 | Ethox TAM-2 DQ | Lithium stearate | 50:10:2 | Two-phase, did not gel |
| 199 | Drakeol 10 | Ethox TAM-2 DQ | Magnesium stearate | 50:10:2 | Two-phase, did not gel |
| 200 | Drakeol 10 | Ethox TAM-2 DQ | Zinc stearate | 50:10:2 | Two-phase, did not gel |
| 201 | Drakeol 10 | | Sodium stearate | 98:2 | Unstable medium clear |
| | | | | | gel |
| 202 | Drakeol 10 | | Sodium stearate | 95:5 | Unstable hard |
| | | | | | translucent gel |
| 203 | Drakeol 10 | | Sodium stearate | 90:10 | Unstable hard opaque |
| | | | | | gel |
| 204 | Drakeol 10 | Ethox TAM-2 DQ | Sodium stearate | 80:16:4 | Hard translucent gel |
| 205 | Drakeol 10 | Ethox CAM-2 | Sodium oxalate | 50:10:2 | Sodium oxalate insoluble |
| | | | | | in Ethox CAM-2 |
| 206 | Drakeol 10 | Ethox CAM-2 | Sodium EDTA | 50:10:2 | Sodium EDTA insoluble |
| | | | | | in Ethox CAM-2 |
| 207 | Drakeol 10 | | Sodium oxalate | 50:2 | Sodium oxalate insoluble |
| | | | | | in Drakeol 10 |
| 208 | Drakeol 10 | | Aluminum stearate 404 | 50:2 | Medium translucent gel |
| 209 | Drakeol 10 | | Aluminum stearate NF | 50:2 | Medium opaque gel |
| 210 | Drakeol 10 | | Calcium stearate | 50:2 | some gelatinous material |
| 211 | Drakeol 10 | | Lithium stearate | 50:2 | Medium opaque gel |
| 212 | Drakeol 10 | | Magnesium stearate | 50:2 | Medium translucent gel |
| 213 | Drakeol 10 | | Zinc stearate | 50:2 | Did not gel |
| 214 | Drakeol 10 | Ethox TAM-2 DQ | Sodium oxalate | 52:10:2 | Two-phase, did not gel |
| 215 | d-Limonene | Ethox 4074 | Sodium 12- | 5:1:0.2 | Soft translucent gel |
| | | | hydroxystearate | | |
| 216 | d-Limonene | Ethox 4074 | Sodium oleate | 5:1:0.2 | Did not gel |
| 217 | d-Limonene | Ethox CAM-2 | Sodium stearate | 5:1:0.2 | Soft translucent gel |
| 218 | Drakeol 10 | Ethox 4074 | Sodium dodecyl sulfate | 50:10:2 | Soft opaque gel |
| 219 | Drakeol 10 | | Sodium dodecyl | 50:10:2 | Did not gel |
| | | | sulfate/sodium stearate | | |
| 220 | Drakeol 10 | Diethylenetriamine | Sodium stearate | 50:10:2 | Did not gel |
| | | pentacetic acid | | | |
| 221 | Drakeol 10 | Ethylenediamine | Sodium stearate | 50:10:2 | Did not gel |
| | | tetraacetic acid | | | |
| 222 | Isopropanol | Ethox 4074 | Sodium stearate/water | 50:10:2:5 | Hard opaque gel |
| 223 | Isopropanol | | Sodium stearate/water | 50:2:5 | Hard opaque gel |
| 224 | Propylene glycol | Ethox CAM-2 DMS | Sodium stearate | 50:10:2 | Did not gel |
| | | quat. 85% in propylene | | | |
| | | glycol | | | |
| 225 | Propylene glycol | Ethox 4074 | Sodium stearate | 50:10:2 | Did not gel |
| 226 | Propylene glycol | Ethox CAM-2 | Sodium stearate | 50:10:2 | Did not gel |
| 227 | Propylene glycol | Ethfac 103 | Sodium stearate | 50:10:2 | Soft opaque gel |
| 228 | Propylene glycol | POP(1.1) laurate | Sodium stearate | 50:10:2 | Soft opaque gel |
| 229 | Propylene glycol | | Sodium stearate | 50:2 | Hard opaque gel |
| 230 | Propylene glycol | E-Sperse 120 (50%) | Sodium stearate | 50:20:2 | Soft opaque gel |
| 231 | Propylene glycol | | Sodium 12- | 50:2 | Did not gel |
| | | | hydroxystearate | | |
| 232 | Propylene glycol | | Sodium oleate | 50:2 | Did not gel |
| 233 | Ethanol | Ethox 4074 | Sodium stearate | 80:16:4 | Repeat of example 156 |
| 234 | d-Limonene | Ethox 4075 | Sodium stearate | 50:45:5 | Repeat of example 108 |
| 235 | Ethox 2156 | Ethox 4074 | Sodium stearate | 70:25:5 | Repeat of example 105 |
| 236 | Soybean oil | Ethox 4074 | Sodium stearate | 90:9:1 | Repeat of example 97 |
| 237 | Water | Ethox 4075 | Sodium stearate | 90:10 | 10% aqueous stability - |
| | | | | | oils out |
| 238 | Water | Example 237 | | | 1% aqueous stability - |
| | | | | | oils out |
| 239 | | Ethox 4074 | Sodium stearate | 300:60 | Soft solid |
| 240 | Drakeol 10 | Example 239 | | 50:12 | Hard opaque gel |
| 241 | Butyl palmitate | Example 239 | | 5:1 | Soft opaque gel |
| 242 | | Ethox 3216 | Sodium stearate | 300:60 | Semi-solid |
| 243 | Drakeol 10 | Ethoxylated sorbitol | Sodium stearate | 50:10:2 | Two-phase, did not gel |
| | | tetraoleate | | | |
| 244 | Drakeol 10 | Ethoxylated sorbitol | Sodium stearate | 50:10:2 | Two-phase, did not gel |
| | | hexaoleate | | | |
| 245 | Drakeol 10 | Glycerol 2- | Sodium stearate | 50:10:2 | Soft opaque gel |
| | | ethylhexanoate | | | |
| 246 | Drakeol 10 | Glycerol 2- | | 50:12 | Did not gel |
| | | ethylhexanoate Na+ | | | |
| 247 | Drakeol 10 | Ethox CAM-2 DMS | Sodium stearate | 50:10:2 | Did not gel |
| 248 | d-Limonene | Glycerol 2- | Sodium stearate | 10:1:0.5 | Soft opaque gel |
| | | ethylhexanoate | | | |
| 249 | Water | Glycerol 2- | Sodium stearate | 90:9:1 | 10% aqueous stability - |
| | | ethylhexanoate | | | oils out |
| 250 | Drakeol 10 | 2-Phenoxyethanol | Sodium stearate | 50:10:2 | Soft opaque gel |
| 251 | Drakeol 10 | Glycerol benzoate | Sodium stearate | 50:10:2 | Two-phase - top layer |
| | | | | | is a soft gel |
| 252 | Xylenes | 2-Phenoxyethanol | Sodium stearate | 50:10:2 | Soft opaque gel |
| 253 | Xylenes | Glycerol benzoate | Sodium stearate | 50:10:2 | Soft opaque gel |
| 254 | Toluene | 2-Phenoxyethanol | Sodium stearate | 50:10:2 | Thick opaque liquid |
| 255 | Toluene | Glycerol benzoate | Sodium stearate | 50:10:2 | Thick opaque liquid |
| 256 | Drakeol 10 | Glycerol benzoate | Sodium stearate | 50:10:2 | Medium opaque gel |
| 257 | Drakeol 10 | SAM-2 DMS quat. 82% | Sodium stearate | 50:10:2 | Soft opaque gel |
| | | in propylene glycol | | | |
| 258 | Drakeol 10 | Ethox 3216 | Sodium stearate | 50:10:2 | Thick opaque liquid |
| 259 | Drakeol 10 | Polyglycerol-3 stearate | Sodium stearate | 50:10:2 | Hard opaque gel |
| 260 | Drakeol 10 | Polyaldo 10-1-S | Sodium stearate | 50:10:2 | Hard opaque gel |
| 261 | Drakeol 10 | Polyglycerol-3 stearate | Sodium stearate | 50:5:2 | Medium opaque gel |
| 262 | Drakeol 10 | Polyglycerol-3 stearate | Sodium stearate | 50:5:1 | Hard opaque gel |
| 263 | Drakeol 10 | Polyglycerol-3 stearate | Sodium stearate | 50:2:2 | Medium opaque gel |
| 264 | Drakeol 10 | C1214 Borate | Sodium stearate | 50:10:2 | Did not gel - borate |
| | | | | | insoluble |
| 265 | Propylene glycol | Polyether quat (DMAPA | Sodium stearate | 50:10:2 | Thick opaque liquid |
| | | lauramide + PEG600 DC) | | | |
| 266 | Propylene glycol | Polyetherquat(DMAPA | Sodium stearate/water | 50:10:2:2.5 | Opaque liquid |
| | | lauramide + PEG600 | | | |
| | | DC) | | | |
| 267 | Drakeol 10 | Polyether quat (DMAPA | Sodium stearate | 50:10:2 | Did not gel |
| | | lauramide + PEG 600 | | | |
| | | DC) | | | |
| 268 | Isopropyl | Ethox 4074 | Sodium stearate | 50:10:2 | Medium opaque gel |
| | myristate | | | | |
| 269 | α-Pinene | Ethox 4075 | Sodium stearate | 8:1.5:0.5 | Soft opaque gel |
| 270 | Ethox ICHD | Ethox 4074 | Sodium stearate | 50:10:2 | Medium opaque gel |
| 271 | α-Pinene | Glycerol sesqui-2- | Sodium stearate | 8:1.5:0.5 | Soft opaque gel |
| | | ethylhexanoate | | | |
| 272 | d-Limonene | Ethox CAM-2 | Sodium stearate | 10:1:0.5 | Hard translucent gel |
| 273 | d-Limonene | Ethox TAM-2 DQ | Sodium stearate | 10:1:0.5 | Soft opaque gel |
| 274 | d-Limonene | Ethox SAM-2 | Sodium stearate | 10:1:0.5 | Soft opaque gel |
| 275 | d-Limonene | Ethox 3331 | Sodium stearate | 10:1:0.5 | Medium opaque gel |
| 276 | d-Limonene | Glycerol formal | Sodium stearate | 10:1:0.5 | Soft opaque gel |
| 277 | d-Limonene | POE(10)glycerol formal | Sodium stearate | 10:1:0.5 | Medium translucent gel |
| 278 | d-Limonene | Ethox CAM-2/ | Sodium stearate | 10:1:1:0.5 | Medium translucent gel |
| | | POP(1.1) laurate | | | |
| 279 | Drakeol 10 | Ethox DT-15 DMS quat | Sodium stearate | 50:10:2 | Did not gel |
| | | 80% in propylene glycol | | | |
| 280 | Drakeol 10 | Tallowpentamethylpropylenediammonoim | Sodium stearate | 50:10:2 | Hard opaque gel |
| | | bismethosulfate | | | |
| 281 | Drakeol 10 | ERS 00212 (50% | Sodium stearate | 50:10:2 | Did not gel |
| | | active) | | | |
| 282 | d-Limonene | Ethox CAM-2 | Sodium stearate | 10:1:1 | Medium opaque gel |
| 283 | d-Limonene | Ethox CAM-2 | Sodium stearate | 10:1:0.25 | Soft translucent gel |
| 284 | d-Limonene | Ethox CAM-2 | Sodium stearate | 10:2:0.5 | Medium translucent gel |
| 285 | Drakeol 10 | Ethox L-61 | Sodium stearate | 50:10:2 | Did not gel |
| 286 | Drakeol 10 | Dimethyl palmitamine | Sodium stearate | 50:10:2 | Medium opaque gel |
| 287 | Drakeol 10 | Dimethyl palmitamine | Sodium stearate | 50:5:1 | Did not gel |
| 288 | Drakeol 10 | Monoethanol amine | Sodium stearate | 50:10:2 | Did not gel |
| 289 | Drakeol 10 | Diethanol amine | Sodium stearate | 50:10:2 | Did not gel |
| 290 | Drakeol 10 | Oleoyl sarcosine | Sodium stearate | 50:10:2 | Clear liquid |
| 291 | Drakeol 10 | Dimethyl stearamine | Sodium stearate | 50:10:2 | Soft gel |
| 292 | Drakeol 10 | 2-Ethylhexylamine | Sodium stearate | 50:10:2 | Soft gel |
| 293 | Drakeol 10 | Isostearic | Sodium stearate | 50:10:2 | Did not gel |
| | | diethanolamide | | | |
| 294 | Drakeol 10 | Dimethyl soyamine | Sodium stearate | 50:10:2 | Did not gel |
| 295 | Drakeol 10 | POE(2)bisphenol A | Sodium stearate | 50:10:2 | Did not gel |
| 296 | | Dimethyl stearamine/ | | 20:25 | Clear liquid |
| | | isostearic acid | | | |
| 297 | Drakeol 10 | Example 296 | Sodium methoxide | 50:10:2 | Did not gel |
| 298 | Drakeol 10 | 1,4-Cyclohexane | Sodium stearate | 50:10:2 | Did not gel |
| | | dimethanol | | | |
| 299 | d-Limonene | 1,4-Cyclohexane | Sodium stearate | 10:1:0.5 | Medium opaque gel |
| | | dimethanol | | | |
| 300 | Drakeol 10 | Glycerol formal | Sodium stearate | 50:10:2 | Two-phase, did not gel |
| 301 | d-Limonene | Sodium phosphate | Sodium stearate | 10:1:0.5 | Sodium phosphate |
| | | monobasic | | | monobasic insoluble |
| 302 | d-Limonene | Sodium phosphate | Sodium stearate | 10:0.5:2:0.5 | Sodium phosphate |
| | | monobasic/water | | | monobasic insoluble |
| 303 | Drakeol 10 | POE(1.1)Tung oil fatty | Sodium stearate | 50:10:2 | Soft opaque gel |
| | | acid | | | |
| 304 | d-Limonene | POE(1.1)Tung oil fatty | Sodium stearate | 10:1:0.5 | Medium opaque gel |
| | | acid | | | |
| 305 | d-Limonene | Sodium laureth sulfate/ | | 5:0.5:0.15:4.35 | White solid with ‘plastic’ |
| | | Ethox HVB/water | | | feel |
| 306 | α-Pinene | | Sodium stearate | 9:1 | Soft opaque gel |
| 307 | d-Limonene | Ethal DA-6/Ethfac | | 2.5:1.5:0.5:0.2:5.5 | Clear liquid |
| | | 353/PPG-460/water | | | |
| 308 | d-Limonene | Ethal DA-6/Ethfac | | 2.5:1.5:0.5:0.2:0.2:5.5 | Clear liquid |
| | | 353/PPG-460/Ethox | | | |
| | | HVB/water | | | |
| 309 | Drakeol 10 | Polyphosphoric acid | Sodium stearate | 50:10:2 | Two-phase, did not gel |
| 310 | d-Limonene | Ethal DA-6/Ethfac | | 5:1.5:0.5:0.2:5.5 | White opaque fluid |
| | | 353/PPG-460/water | | | |
| 311 | Drakeol 10 | Lecithin | Sodium stearate | 50:10:2 | Viscous opaque liquid |
| 312 | Drakeol 10 | 1H-Imidazolium, 1- | Sodium stearate | 50:10:2 | 2 phase soft gel |
| | | ethyl-2-(8- | | | |
| | | heptadecenyl)-4,5- | | | |
| | | dihydro-3-(2- | | | |
| | | hydroxyethyl)-, ethyl | | | |
| | | sulfate (salt) | | | |
| 313 | Drakeol 10 | Coco-ethyldimonium | Sodium stearate | 50:10:2 | 2 phase did not gel |
| | | ethosulfate | | | |
| 314 | Drakeol 10 | 1-Hexadecanaminium, | Sodium stearate | 50:10:2 | Creamy soft opaque gel |
| | | N,N-dihexadecyl-N- | | | |
| | | methyl-, chloride | | | |
| 315 | d-Limonene | Ethox CAM-2 | Sodium stearate | 9:0.67:0.33 | Medium opaque gel |
| 316 | d-Limonene | Ethox CAM-2 | Sodium stearate | 10:1:0.5 | Medium opaque gel |
| 317 | d-Limonene | Ethox HVB | Sodium laureth | 5:0.5:0.5:4.35 | Soft opaque gel |
| | | | sulfate/water | | |
| 318 | Drakeol 10/ | Ethox 4074 | Sodium stearate | 10:10:2:0.3 | Soft opaque gel |
| | d-limonene | | | | |
| 319 | d-Limonene/ | Ethox DA-6/Ethfac | | 4:4:1.4:0.4:0.2 | Opaque liquid/splits |
| | Water | 353/PPG-460 | | | |
| 320 | d-Limonene/ | Ethox DA-6/Ethfac | | 5:11:3:1:0.4 | Repeat of example 307 |
| | Water | 353/PPG-460 | | | |
| 321 | d-Limonene/ | | Sodium stearate | 8.5:1:0.5 | Hard opaque gel |
| | Water | | | | |
| 322 | d-Limonene/ | Ethox DA-6/Ethfac | | 4:4:1.4:0.4:0.2 | Boil over |
| | Water | 353/PPG-460 | | | |
| 323 | d-Limonene/ | Ethox HVB | Sodium laureth sulfate | 5:4.35:1:0.5 | Thick material - too |
| | Water | | | | much Ethox HVB |
| 324 | | Ethox DA-6/Ethfac | | 30:10:4 | Clear liquid - strong odor |
| | | 353/PPG-460 | | | of Decyl alcohol |
| 325 | Example 324 | Ethox HVB | | 18:2 | Solidifies upon cooling |
| 326 | | Ethox MI-9/Ethfac | | 30:10:4 | Clear one phase - no |
| | | 353/PPG-460 | | | odor |
| 327 | d-Limonene/ | Example 326 | | 5:11:4.4 | Opaque thick cream |
| | Water | | | | |
| 328 | d-Limonene/ | Example 326 | | 5:11:4.4 | Soft opaque cream |
| | Water | | | | |
| 329 | d-Limonene/ | Ethox BA- | | 5:5:5:1 | Soft opaque cream |
| | Water | 25/25%/Ethfac 353 | | | |
| 330 | d-Limonene/ | Example 324 | | 5:11:4.4 | Hazy liquid |
| | Water | | | | |
| 331 | | POE(7)decyl alcohol/ | | 30:10:4 | Clear liquid |
| | | Ethfac 353/PPG 460 | | | |
| 332 | d-Limonene/ | Example 331 | | 5:11:4.4 | Hazy liquid |
| | Water | | | | |
| 333 | Drakeol 10 | Ethox 4075 | Sodium stearate | 100:6:0.6 | Soft solid |
| 334 | Drakeol 10 | Ethox 4074 | Sodium stearate | 100:6:0.6 | Clear soft gel |
| 335 | Drakeol 10 | Ethox 4074 | Sodium stearate | 100:20:2 | Medium opaque gel |
| 336 | Cyclomethicone + | Isopropanol/water | Sodium stearate | 10:2:2:2 | Hard opaque gel |
| | Octyl | | | | |
| | dimethicone | | | | |
| | ethoxy glucoside | | | | |
| 337 | 20 cst Silicone oil | Example 336 | | 50:10 | Hard opaque gel |
| 338 | Cyclomethicone + | Isopropanol/water | Sodium stearate | 10:1:1:1 | Hard translucent gel |
| | Octyl | | | | |
| | dimethicone | | | | |
| | ethoxy glucoside | | | | |
| 339 | Cyclomethicone + | Ethox 4075 | Sodium stearate | 10:2:1 | Hard translucent gel |
| | Octyl | | | | |
| | dimethicone | | | | |
| | ethoxy glucoside | | | | |
| 340 | Cyclomethicone + | Ethox 4075 | Sodium stearate | 10:1:0.25 | Clear hard gel |
| | Octyl | | | | |
| | dimethicone | | | | |
| | ethoxy glucoside | | | | |
| 341 | 20 cst Silicone oil | Example 339 | | 50:all | Insoluble |
| 342 | Drakeol 10 | Ethox 4074 | Sodium stearate | 100:6:0.6 | Hard clear gel |
| 343 | Drakeol 10 | Ethox 4075 | Sodium stearate | 100:6:0.6 | Hard clear gel |
| 344 | Drakeol 10 | Glycerol distearate | Sodium stearate | 50:10:2 | Medium opaque gel |
| 345 | Drakeol 10 | Glycerol dimyristate | Sodium stearate | 50:10:2 | Medium opaque gel |
| 346 | Functionalized | Ethox 4075 | Sodium stearate | 10:1:0.25 | Medium translucent gel |
| | silicone | | | | |
| 347 | Functionalized | Ethox 4075 | Sodium stearate | 10:1:0.25 | Medium translucent gel |
| | silicone | | | | |
| 348 | Functionalized | Ethox 4075/isopropanol | Sodium stearate | 10:1:0.25 | Medium translucent gel |
| | silicone | | | | |
| 349 | Functionalized | Ethox 4075 | Sodium stearate | 50:10:2 | Medium opaque gel |
| | silicone | | | | |
| 350 | Functionalized | Ethox CAM-2 | Sodium stearate | 50:10:2 | Medium opaque gel |
| | silicone | | | | |
| 351 | Drakeol 10 | Ethox 4075 | Sodium stearate | 500:30:3 | Clear soft gel |
| 352 | Isopropanol | Ethox 4075/water | Sodium stearate | 50:5:5:2 | Hard translucent gel |
| 353 | Isopropanol | Water | Sodium stearate | 50:5:2 | Hard translucent gel |
| 354 | Isopropanol | Ethox 4075/water | Sodium stearate | 50:10:5:2 | Hard translucent gel |
| 355 | Isopropanol | Ethox 4075/water | Sodium stearate | 50:10:10:2 | Hard translucent gel |
| 356 | Drakeol 10 | Ethox 4075 | Sodium stearate | 500:100:10 | Hard clear gel |
| 357 | Isopropanol | Ethox 4075/water | Sodium stearate | 500:50:50:10 | Hard translucent gel |
| 358 | Soybean oil | Ethox 4075 | Sodium stearate | 50:20:2 | Hard clear gel |
| 359 | Soybean oil | Ethox 4075 | Sodium stearate | 400:160:16 | Hard clear gel |
| 360 | Isopropanol | Ethox 4074/water | Sodium stearate | 50:10:10:2 | Hard translucent gel |
| 361 | Isopropanol | Ethox 4074/water | Sodium stearate | 400:80:80:16 | Hard translucent gel |
| 362 | Ethox 2156 | Ethox 4075 | Sodium stearate | 50:20:2 | Hard opaque gel |
| 363 | Isopropanol | Ethox 4075/water | Sodium stearate | 400:80:80:16 | Hard translucent gel |
| 364 | d-Limonene | Ethox 4075 | Sodium stearate | 10:3:0.5 | Some gel |
| 365 | d-Limonene | Glycerol sesqui-2- | Sodium stearate | 10:3:0.5 | Soft opaque gel |
| | | ethylhexanoate | | | |
| 366 | Cyclomethicone + | Ethox 4075 | Sodium stearate | 50:5:1.25 | Hard opaque gel |
| | Octyl | | | | |
| | dimethicone | | | | |
| | ethoxy glucoside | | | | |
| 367 | Isopropanol | POE(12) glycerol | Sodium stearate | 50:10:10:2 | Hard translucent gel |
| | | dimyristate/water | | | |
| 368 | Isopropanol | ERS 00206/water | Sodium stearate | 50:10:10:2 | Hard translucent gel |
| 369 | Isopropanol | POE(23) glycerol | Sodium stearate | 50:10:10:2 | Hard translucent gel |
| | | dimyristate/water | | | |
| 370 | Cyclomethicone + | Ethox 4075 | Sodium stearate | 100:10:2.5 | Hard opaque gel |
| | Octyl | | | | |
| | dimethicone | | | | |
| | ethoxy glucoside | | | | |
| 371 | Isopropanol | POE(12) glycerol | Sodium stearate | 50:10:5:2 | Some gel |
| | | dimyristate | | | |
| 372 | Cyclomethicone + | Ethox 4075 | Sodium stearate | 50:10:1.25 | Hard opaque gel |
| | Octyl | | | | |
| | dimethicone | | | | |
| | ethoxy glucoside | | | | |
| 373 | Isopropanol | Ethox ML-5/water | Sodium stearate | 50:10:10:2 | Hard translucent gel |
| 374 | Isopropanol | POP(5) CFA/water | Sodium stearate | 50:10:10:2 | Hard translucent gel |
| 375 | Isopropanol | Ethox CO-5/water | Sodium stearate | 50:10:10:2 | Hard translucent gel |
| 376 | Isopropanol | water | Sodium stearate | 50:10:2 | Hard translucent gel |
| 377 | Isopropanol | POE(3) TMP/water | Sodium stearate | 50:10:10:2 | Hard translucent gel |
| 378 | Drakeol 10 | Ethox 4075 | Sodium oleate/sodium | 50:10:1:1 | Hard opaque gel |
| | | | stearate | | |
| 379 | Drakeol 10 | CHC015-053 | | 25:25:2 | Hard opaque gel |
| 380 | Drakeol 10 | CHC015-053 | Sodium stearate | 46:6 | Soft opaque gel |
| 381 | Drakeol 10 | CHC015-060 | | 45:10 | Hazy liquid |
| 382 | Drakeol 10 | CHC015-057 | | 45:10 | Hazy liquid |
| 383 | Drakeol 10 | CHC015-060 | Sodium stearate | 45:10:2 | Hazy liquid |
| 384 | Propylene glycol | CHC015-060 | | 45:10 | 2-layers |
| 385 | Drakeol 10 | CHC015-064 | | 45:10 | Thick opaque liquid |
| 386 | Drakeol 10 | CHC015-065 | Sodium stearate | 50:10:2 | Clear liquid |
| 387 | Example 581 | CHC015-065 | Sodium stearate | 50:10:2 | Hard solid |
| 388 | Drakeol 10 | Oleoyl sarcosine | Sodium stearate | 50:10:2 | Hazy liquid |
| 389 | Example 581 | CHC015-065 | Sodium stearate/water | 50:10:2.6:10 | 2-layers |
| 390 | Example 581 | CHC015-065 | Sodium | 50:10:1:10 | 2-layers |
| | | | hydroxide/water | | |
| 391 | Drakeol 7 | Ethox 4075 | Sodium stearate | 50:10:1 | Medium clear gel |
| 392 | Drakeol 7 | Ethox 4075 | Sodium stearate | 50:10:2 | Hard opaque gel |
| 393 | Drakeol 7 | Ethox 4075 | Sodium stearate | 50:5:1 | Hard opaque gel |
| 394 | Drakeol 7 | Ethox 4075 | Sodium stearate | 50:5:0.5 | Medium clear gel |
| 395 | Drakeol 10 | CHC015-071 | Sodium stearate | 50:10:2 | Did not gel |
| 396 | Example 581 | CHC015-071 | Sodium stearate/ | 50:10:2:5 | Soft opaque gel, some |
| | | | Ammonium hydroxide | | ppt |
| 397 | Example 581 | CHC015-071 | Sodium stearate/ | 50:10:2:2 | 2-phase |
| | | | Ammonium hydroxide | | |
| 398 | CHC015-071 | Ammonium hydroxide | Water | 10:2:24 | 2-phase |
| 399 | CHC015-071 | Sodium hydroxide | | 10:2:2 | Viscous opaque paste |
| 400 | Drakeol 10 | ERS 00208 | Sodium stearate | 50:10:2 | White ppt |
| 401 | Drakeol 10 | CHC015-071 DETA | Sodium stearate | 50:10:2 | Liquid |
| 402 | Example 581 | CHC015-071 DETA | | 50:10 | 2-phase |
| 403 | Example 581 | CHC015-071 MEA | | 50:10 | 2-phase |
| 404 | Drakeol 10 | CHC015-071 MEA | | 50:10 | 2-phase |
| 405 | Example 581 | CHC015-071 DEA | | 50:10 | 2-phase |
| 406 | Drakeol 10 | CHC015-071 DEA | | 50:10 | 2-phase |
| 407 | Example 581 | CHC015-071 TEA | | 50:10 | 2-phase |
| 408 | Drakeol 10 | CHC015-071 TEA | | 50:10 | 2-phase |
| 409 | Drakeol 10 | CHC015-071 MEA | Sodium stearate | 50:10:2 | Thick opaque liquid |
| 410 | Example 581 | CHC015-071 MEA | Sodium stearate | 50:10:2 | Soft opaque gel |
| 411 | Drakeol 10 | Ethal 326/Ethox HCO- | Sodium stearate/water | 40:20:20:15:2:3 | 2-phase |
| | | 40/PEG 400 | | | |
| 412 | Drakeol 10 | Ethal 326/Ethox | Sodium stearate/water | 40:20:20:15 | 2-phase |
| | | HCO-40/PEG 401 | | | |
| 413 | Glycol ether EB | | Sodium stearate | 50:2 | Soft opaque gel |
| 414 | Glycol ether EB | Sodium stearate | Water | 50:2:5 | soft opaque gel |
| 415 | Glycol ether EB | Ethox 4075 | Sodium stearate | 50:10:2 | Did not gel |
| 416 | Example 414 | Example 415 | | 25:25 | Did not gel |
| 417 | Glycol ether EB | Ethox 4075 | Sodium stearate/water | 50:5:5:1 | Clear liquid |
| 418 | Glycol ether EB | Ethox CAM-2 | Sodium stearate | 50:10:2 | Soft opaque gel |
| 419 | Glycol ether EB | Ethox TAM-2 dq | Sodium stearate | 50:10:2 | Clear liquid |
| 420 | Glycol ether PnP | Ethox 4075 | Sodium stearate | 50:10:2 | Medium opaque gel |
| 421 | Glycol ether PM | Ethox 4075 | Sodium stearate | 50:10:2 | Medium opaque gel |
| 422 | Glycol ether | Ethox 4075 | Sodium stearate | 50:10:2 | Medium opaque gel |
| | TPnB | | | | |
| 423 | Glycol ether DB | Ethox 4075 | Sodium stearate | 50:10:2 | Thickened liquid |
| 424 | Glycol ether EPh | Ethox 4075 | Sodium stearate | 50:10:2 | Clear hard gel |
| 425 | Glycol ether DM | Ethox 4075 | Sodium stearate | 50:10:2 | Hard opaque gel |
| 426 | Glycol ether EPh | Ethox 4075 | Sodium stearate/water | 50:10:2:5 | Hard hazy gel |
| 427 | Glycol ether EPh | | Sodium stearate | 50:2 | Hard opaque gel |
| 428 | Glycol ether EPh | Ethox 4075 | Sodium stearate | 100:20:4 | Hard clear gel |
| 429 | Drakeol 10 | Octyl/decyl sesquicitrate | Sodium stearate | 50:10:2 | Liquid |
| 430 | Drakeol 10 | | Octyl/decyl citrate Na+ | 50:10 | Clear liquid |
| 431 | Hydrogenated | Ethox 4075 | Sodium stearate | 50:10:1 | Hard clear gel |
| | polydecene | | | | |
| 432 | Isopropanol | Ethox 4075/Ethylene | Sodium stearate | 50:10:5:1 | Soft translucent gel |
| | | glycol | | | |
| 433 | DiTMP | Ethox 4075 | Sodium stearate | 50:10:1 | Hazy liquid |
| | tetrabenzoate | | | | |
| 434 | Propylene glycol | Water/Ethal LA-4/Ethal | Sodium stearate | 40:5:2:2:10:1 | Liquid |
| | | LA-23/Ethox 4075 | | | |
| 435 | 20 cst Silicone oil | Cyclomethicone + Octyl | Sodium stearate | 50:10:2:0.5 | Some solids - flowable |
| | | dimethicone ethoxy | | | |
| | | glucoside/Ethox 4075 | | | |
| 436 | 20 cst Silicone oil | Cyclomethicone + Octyl | Sodium stearate | 50:10:2:2:5:1 | 2-layers |
| | | dimethicone ethoxy | | | |
| | | glucoside Isopropanol/ | | | |
| | | water/Ethox 4075 | | | |
| 437 | 20 cst Silicone oil | Cyclomethicone + Octyl | Sodium stearate | 50:10:5:5:10:1 | 2-layers |
| | | dimethicone ethoxy | | | |
| | | glucoside Isopropanol/ | | | |
| | | water/Ethox 4075 | | | |
| 438 | 20 cst Silicone oil | Functionalized silicone/ | Sodium stearate | 50:10:10:2 | 2-layers |
| | | Ethox 4075 | | | |
| 439 | 20 cst Silicone oil | Functionalized silicone/ | Sodium stearate | 50:10:10:2 | 2-layers |
| | | Ethox 4075 | | | |
| 440 | 20 cst Silicone oil | Functionalized silicone/ | Sodium stearate | 50:10:10:10:2 | Soft opaque gel |
| | | Ethox 4067/Ethox 4075 | | | |
| 441 | Drakeol 10 | Ethox 4075 | Sodium oleate | 100:20:4 | Clear liquid |
| 442 | 20 cst Silicone oil | POP(8) decyl/Ethox | Sodium stearate | 50:10:10:2 | 2-layers |
| | | 4075 | | | |
| 443 | POP(8) decyl | Ethox 4075 | Sodium stearate | 50:10:2 | Medium opaque gel |
| | alcohol | | | | |
| 444 | Ethal 326 | Ethox 4075 | Sodium stearate | 50:10:2 | Hard opaque gel |
| 445 | Ethfac 103 | Ethox 4075 | Sodium stearate | 50:10:2 | Clear liquid |
| 446 | Ethfac 104 | Ethox 4075 | Sodium stearate | 50:10:2 | Clear liquid |
| 447 | 20 cst Silicone oil | Ethal TDA-3/Ethox 4075 | Sodium stearate | 50:5:10:2 | 2-phase |
| 448 | Hydrogenated | Ethox 4075 | Sodium stearate | 50:10:2 | Hard opaque gel |
| | polydecene | | | | |
| 449 | Hydrogenated | Ethox 4075 | Sodium stearate | 100:20:2 | Hard clear gel |
| | polydecene | | | | |
| 450 | 20 cst Silicone oil | Isopropanol/water/ | Sodium stearate | 50:5:5:10:10:2 | 2-phase |
| | | POP(8)decyl/Ethox 4075 | | | |
| 451 | Hydrogenated | Ethal 326/Ethox 4075 | Sodium stearate | 50:3:7:1 | Hard clear gel |
| | polydecene | | | | |
| 452 | Dow Corning | Ethox 4074 | Sodium stearate | 50:10:2 | Medium opaque gel |
| | 5562 | | | | |
| 453 | Drakeol 10 | Dendritic polymer | Sodium stearate | 50:10:2 | 2-layers |
| 454 | Drakeol 10 | Glycerol | Sodium stearate | 50:10:2 | Soft gel |
| | | monoisostearate | | | |
| 455 | Drakeol 10 | Glycerol | Sodium stearate | 50:10:1 | Medium opaque gel |
| | | monoisostearate | | | |
| 456 | Drakeol 10 | POE(2.2) dimerate | Sodium stearate | 50:10:2 | Medium opaque gel |
| 457 | Drakeol 10 | POE(2.2) dimerate | Sodium stearate | 50:10:1 | Thickened liquid |
| 458 | Drakeol 10 | POP(2.2) dimerate | Sodium stearate | 50:10:2 | Thickened opaque liquid |
| 459 | Drakeol 10 | DiTPnB azelate | Sodium stearate | 50:10:2 | Thickened opaque liquid |
| 460 | Drakeol 10 | POE(23) glycerol | Sodium stearate | 50:10:2 | 2-layers |
| | | dioleate | | | |
| 461 | Drakeol 10 | Ethox 4074/Glycerol | Sodium stearate | 50:7.5:2.5:2 | Hard opaque gel |
| | | monoisostearate | | | |
| 462 | Drakeol 10 | Ethox 4074/Glycerol | Sodium stearate | 50:7.5:2.5:1 | Hard clear gel |
| | | monoisostearate | | | |
| 463 | Drakeol 10 | Ethox 4075/water | Sodium stearate | 200:40:4:8 | Hard hazy gel |
| 464 | Drakeol 10 | Coco imidazoline | Sodium stearate | 50:10:2 | Soft opaque gel |
| 465 | Soybean oil | POP(200) isolignoceryl | | 50:5 | Clear liquid |
| | | alcohol | | | |
| 466 | Propylene glycol | ERS 00211 | Sodium stearate | 50:10:2 | Did not gel |
| 467 | Propylene glycol | Water | Sodium stearate | 50:10:2 | Hard opaque gel |
| 468 | Propylene glycol | POE(200) isolignoceryl | | 50:1 | Soft opaque gel |
| | | behenate | | | |
| 469 | Propylene glycol | POE(200) isolignoceryl | | 50:5 | Hard opaque gel |
| | | behenate | | | |
| 470 | Propylene glycol | POE(200) isolignoceryl | Sodium stearate | 50:1:10:2 | Soft opaque gel |
| | | behenate/Ethox 4075 | | | |
| 471 | Propylene glycol | POP(2.2) dimerate | Sodium stearate | 50:10:2 | Clear liquid |
| 472 | Example 470 | Water | | All:10 | 2-layers |
| 473 | Example 471 | Water | | All:10 | Clear liquid |
| 474 | Propylene glycol | POP(2.2) dimerate/ | Sodium stearate | 50:10:1:10:2 | 2-layers |
| | | NaCl/Water | | | |
| 475 | Propylene glycol | Ethox 4075/NaCl/Water | Sodium stearate | 50:10:1:10:2 | 2-layers |
| 476 | Propylene glycol | Ethox 4075/NaCl | Sodium stearate | 50:10:1:2 | Sodium chloride is |
| | | | | | insoluble |
| 477 | Propylene glycol | Ethox 4075/NaCl/ | Sodium stearate | 50:10:1:10:2 | 2-phase |
| | | glycerin | | | |
| 478 | Meadowfoam | Ethox 4075 | Sodium stearate | 10:2:0.4 | Hard opaque gel |
| | seed oil | | | | |
| 479 | Meadowfoam | Ethox 4075 | Sodium stearate | 10:2:0.2 | Hard clear gel |
| | seed oil | | | | |
| 480 | Propylene glycol | POP(2.2) dimerate/NaCl | Sodium stearate | 50:10:0.5:2 | Medium opaque gel |
| 481 | Ethox 2156 | Ethox 4075 | Sodium stearate | 50:30:3 | Very hard opaque gel |
| 482 | Ethox 2156 | Ethox 4075 | Sodium stearate | 50:50:4 | Very hard opaque gel |
| 483 | Ethox 2156 | Ethox 4075 | Sodium stearate | 50:10:2 | Medium opaque gel |
| 484 | Ethox 2156 | Ethox 4075 | Sodium stearate | 50:20:2 | Hard opaque gel |
| 485 | Ethox 2156 | Ethox 4075 | Sodium stearate | 50:20:4 | Hard opaque gel |
| 486 | Ethox 2156 | Ethox 4075 | Sodium stearate | 50:5:1 | Soft opaque gel |
| 487 | Ethox 2156 | Ethox 4074 | Sodium stearate | 50:5:1 | Soft opaque gel |
| 488 | Ethox 2156 | Ethox 4075 | Sodium stearate | 50:10:3 | Medium opaque gel |
| 489 | Ethox 2156 | | Sodium stearate | 50:2 | Insoluble |
| 490 | Ethox 2156 | Ethox 4075 | Sodium stearate | 50:20:2 | Soft opaque gel |
| 491 | Ethox 2156 | Ethox 4075 | Sodium stearate | 200:80:8 | Hard opaque gel |
| 492 | Drakeol 10 | Ethox 4075 | Sodium stearate | 50:10:2 | Hard opaque gel |
| 493 | Drakeol 10 | Ethox 4075 | Sodium stearate | 50:10:1 | Hard clear gel |
| 494 | Drakeol 10 | Ethox 4075/EDTA | Sodium stearate | 50:10:1:2 | EDTA insoluble |
| 495 | Drakeol 10 | Ethox 4067/Ethox 4075 | Sodium stearate | 25:10:10:2 | Hard opaque gel |
| 496 | Drakeol 10 | Ethox 4075/ | Sodium stearate | 50:7.5:2.5:2 | Hard opaque gel |
| | | Ethox CAM-2 | | | |
| 497 | Drakeol 10 | Ethox 4075/Ethal 326 | Sodium stearate | 50:7.5:2.5:2 | Hard opaque gel |
| 498 | Drakeol 10 | Ethox 4075/ | Sodium stearate | 50:10:1:5:2 | EDTA insoluble |
| | | EDTA/water | | | |
| 499 | Water | Ethox 4075 | Sodium stearate | 50:10:2 | Thickened opaque liquid |
| 500 | Water | Ethox 4075 | Sodium stearate | 50:10:2 | Thickened opaque liquid |
| 501 | Propylene glycol | Octyl/decyl-2- | I-24(200) behenate | 50:5:5 | Soft opaque gel |
| | | ethylhexanoate | | | |
| 502 | Example 581 | Octyl/decyl-2- | I-24(200) behenate | 50:5:5 | Did not gel |
| | | ethylhexanoate | | | |
| 503 | Propylene glycol | Ethal 326 | I-24(200) behenate | 50:5:5 | Soft opaque gel |
| 504 | Propylene glycol | Octyl/decyl-2- | Sodium stearate | 50:10:2 | Soft opaque gel |
| | | ethylhexanoate | | | |
| 505 | Propylene glycol | Ethal 326 | Sodium stearate | 50:10:2 | Hazy liquid |
| 506 | Propylene glycol | POP(2) dimerate | Sodium stearate | 50:5:2 | 2-layers, soft gel |
| 507 | Propylene glycol | POP(2) dimerate/Ethal | Sodium stearate | 50:2:2:2 | Hard translucent gel |
| | | 326 | | | |
| 508 | Drakeol 10 | Sodium cocoyl | Sodium stearate | 25:5:1 | Insoluble |
| | | hydrolyzed soy protein | | | |
| | | (and) sorbitol | | | |
| 509 | Propylene glycol | Sodium cocoyl | Sodium stearate | 25:5:1 | Hard opaque gel |
| | | hydrolyzed soy protein | | | |
| | | (and) sorbitol | | | |
| 510 | Propylene glycol | Potassium cocoyl | Sodium stearate | 25:5:1 | Medium opaque gel |
| | | hydrolyzed collagen | | | |
| 511 | Propylene glycol | POP(2) dimerate/Ethal | Sodium stearate | 50:1:1:2 | Hard translucent gel |
| | | 326 | | | |
| 512 | Propylene glycol | POP(200) isolignoceryl | | 50:5 | Insoluble |
| | | alcohol | | | |
| 513 | Drakeol 10 | POP(200) isolignoceryl | | 50:5 | 2-phase |
| | | alcohol | | | |
| 514 | Drakeol 10 | POP(200) isolignoceryl | Sodium stearate | 50:10:2 | 2-phase |
| | | alcohol | | | |
| 515 | Propylene glycol | Ethox 4075/Ethal 326 | Sodium stearate | 50:2:2:2 | Hard translucent gel |
| 516 | Propylene glycol | Ethox 4074/Ethal 326 | Sodium stearate | 50:2:2:2 | Hard translucent gel |
| 517 | Propylene glycol | Ethox 4074/Ethal | Sodium stearate | 50:2:2:5:2 | Hard opaque gel |
| | | 326/Water | | | |
| 518 | Drakeol 10 | Ethox 4075 | Sodium stearate | 50:10:2 | Medium translucent gel |
| 519 | Drakeol 10 | Ethox 4075 | Sodium stearate | 50:10:1 | Medium translucent gel |
| 520 | Drakeol 10 | Ethox 4075 | Sodium stearate | 50:10:2 | Medium opaque gel |
| 521 | Drakeol 10 | POP(200) isolignoceryl | | 50:5 | Insoluble |
| | | dimerate | | | |
| 522 | 20 cst Silicone oil | POP(200) isolignoceryl | | 50:5 | Insoluble |
| | | dimerate | | | |
| 523 | Example 581 | POP(200) isolignoceryl | | 50:5 | Insoluble |
| | | dimerate | | | |
| 524 | Drakeol 10 | POP(200) isolignoceryl | | 50:5 | Insoluble |
| | | isostearate | | | |
| 525 | 20 cst Silicone oil | POP(200) isolignoceryl | | 50:5 | Insoluble |
| | | isostearate | | | |
| 526 | Example 581 | POP(200) isolignoceryl | | 50:5 | Insoluble |
| | | isostearate | | | |
| 527 | Propylene glycol | POP(2) dimerate/ | Sodium stearate | 50:2:2:2 | Hard translucent gel |
| | | POP(2.6) Epal 1214 | | | |
| 528 | Propylene glycol | POP(2) dimerate/ | Sodium stearate | 50:2:2:5:0.5:2 | Hard opaque gel |
| | | POP(2.6) Epal 1214/ | | | |
| | | Water | | | |
| 529 | Propylene glycol | POP(2) dimerate/Di- | Sodium stearate | 50:2:2:2 | Soft opaque gel |
| | | TPnB azelate | | | |
| 530 | Propylene glycol | POP(2) dimerate/POP | Sodium stearate | 50:2:2:5:0.5:2 | Hard opaque gel |
| | | (2.6) Epal 1214/Water | | | |
| 531 | Propylene glycol | Ethox 4075/POP(2.2) | Sodium stearate | 50:5:2:2 | 2-layer |
| | | dimerate | | | |
| 532 | Propylene glycol | Ethox 4075 | Calcium stearate | 50:10:2 | Calcium stearate not |
| | | | | | soluble |
| 533 | Propylene glycol | Ethal 326 | Calcium stearate | 50:10:2 | Soft opaque gel |
| 534 | Propylene glycol | Ethal 326/POP(200) | Sodium stearate | 50:5:10:2 | 2-phase |
| | | isolignoceryl isostearate | | | |
| 535 | 20 cst Silicone oil | Cyclomethicone + | | 50:10:10 | Did not gel |
| | | Octyl dimethicone | | | |
| | | ethoxy glucoside/ | | | |
| | | POP(200)I-24 isostearate | | | |
| 536 | Drakeol 10 | Ethox 4075/water | Sodium stearate | 50:10:1:1 | Did not gel |
| 537 | Drakeol 10 | Ethox 4075/water | Sodium stearate | 50:10:1:2 | Hard clear gel |
| 538 | Drakeol 10 | Ethox 4075/water | Sodium stearate | 50:10:2 | Hard opaque gel |
| 539 | Drakeol 10 | Ethox 4075/water | Sodium stearate | 50:10:1:2 | Hard clear gel |
| 540 | Drakeol 10 | Ethox 4075/water | Sodium stearate | 50:10:2:2 | Medium opaque gel |
| 541 | Drakeol 10 | Ethox 4075/water | Sodium stearate | 50:10:5:2 | Soft opaque gel |
| 542 | Drakeol 10 | Ethox 4075/ethylene | Sodium stearate | 50:10:1:2 | Hard clear gel |
| | | glycol | | | |
| 543 | Drakeol 10 | Ethox 4075/propylene | Sodium stearate | 50:10:1:2 | Hard clear gel |
| | | glycol | | | |
| 544 | Drakeol 10 | Ethox 4075/propylene | Sodium stearate | 50:10:2:2 | Very hard translucent gel |
| | | glycol | | | |
| 545 | Drakeol 10 | Ethox 4075/propylene | Sodium stearate | 50:10:1:1:2 | Very hard clear gel |
| | | glycol/Octyl/decyl-2- | | | |
| | | ethyl hexanoate | | | |
| 546 | Drakeol 10 | Ethox 4075/Example | Sodium stearate | 50:10:1:2 | Hard translucent gel |
| | | 581 | | | |
| 547 | Drakeol 10 | Ethox 4075/Ethal 326 | Sodium stearate | 50:10:1:2 | Hard opaque gel |
| 548 | Drakeol 10 | Ethox 4075/POP(2) cetyl | Sodium stearate | 50:10:1:2 | Hard opaque gel |
| | | alcohol | | | |
| 549 | Drakeol 10 | POP(2) cetyl alcohol | Sodium stearate | 50:10:2 | Soft opaque gel |
| 550 | Drakeol 10 | POP(4)POE(1) cetyl | Sodium stearate | 50:10:2 | 2-phase |
| | | alcohol | | | |
| 551 | Drakeol 10 | Ethox 4075/hexane diol | Sodium stearate | 50:10:1:2 | Hard opaque gel |
| 552 | Drakeol 10 | Ethox 4075/Glycerol | Sodium stearate | 50:10:1:2 | Hard opaque gel |
| | | monoisostearate | | | |
| 553 | Drakeol 10 | Pentaerythritol | Sodium stearate | 50:10:2 | Did not gel |
| | | hemipentacocoate | | | |
| 554 | d-limonene | Ethox 4075/water | Sodium stearate | 7.5:1.5:0.5:0.15 | 2-layers |
| 555 | d-limonene | Ethox 4075/water | Sodium stearate | 8:2:0.2:0.2 | 2-layers |
| 556 | d-limonene | Ethox 4075/propylene | Sodium stearate | 8:2:0.2:0.2 | Clear liquid |
| | | glycol | | | |
| 557 | d-limonene | Ethox 4075/propylene | Sodium stearate | 8:2:0.5:1 | Hard opaque gel |
| | | glycol | | | |
| 558 | d-limonene | Propylene glycol | Sodium stearate | 8:3:2 | Very hard opaque gel |
| 559 | d-limonene | Propylene glycol | Sodium stearate | 8:1.5:1 | Very hard opaque gel |
| 560 | d-limonene | Propylene glycol | Sodium stearate | 9:0.75:0.25 | Soft opaque gel |
| 561 | d-limonene | Ethox 4075/propylene | Sodium stearate | 8:0.5:1:1 | Hard opaque gel |
| | | glycol | | | |
| 562 | d-limonene | Ethox 4075/propylene | Sodium stearate | 8:0.5:1:0.5 | Hard opaque gel |
| | | glycol | | | |
| 563 | d-limonene | Glyceryl sesqui-2- | Sodium stearate | 8:0.75:0.75:0.5 | Hard opaque gel |
| | | ethylhexanoate/ | | | |
| | | propylene glycol | | | |
| 564 | d-limonene | Glyceryl sesqui-2- | Sodium stearate | 8:0.75:0.75:0.5 | Hard opaque gel |
| | | ethylhexanoate/ | | | |
| | | propylene glycol | | | |
| 565 | d-limonene | Glyceryl sesqui-2- | Sodium stearate | 8:1:0.5:0.5 | Hard opaque gel |
| | | ethylhexanoate/ | | | |
| | | propylene glycol | | | |
| 566 | d-limonene | Glyceryl sesqui-2- | Sodium stearate | 8.2:0.75:0.75:0.3 | Soft opaque gel |
| | | ethylhexanoate/ | | | |
| | | propylene glycol | | | |
| 567 | d-limonene | Glyceryl sesqui-2- | Sodium stearate | 8.1:0.75:0.75:0.4 | Medium translucent gel |
| | | ethylhexanoate/ | | | |
| | | propylene glycol | | | |
| 568 | d-limonene | Glyceryl sesqui-2- | Sodium stearate | 8:0.75:0.75:0.5 | Hard translucent gel |
| | | ethylhexanoate/glycerin | | | |
| 569 | d-limonene | Glyceryl sesqui-2- | Sodium stearate | 8.2:1:0.4:0.4 | Hard translucent gel |
| | | ethylhexanoate/glycerin | | | |
| 570 | d-limonene | Glyceryl sesqui-2- | Sodium stearate | 8.2:0.4:1:0.4 | 2-phase |
| | | ethylhexanoate/glycerin | | | |
| 571 | d-limonene | Glyceryl sesqui-2- | Sodium stearate | 8:0.75:0.75:0.5 | Hard opaque gel |
| | | ethylhexanoate/ | | | |
| | | diglycerol | | | |
| 572 | d-limonene | Glyceryl sesqui-2- | Sodium stearate | 8:0.75:0.75:0.5 | Soft opaque gel |
| | | ethylhexanoate/ | | | |
| | | di(propylene glycol) | | | |
| 573 | d-limonene | Glyceryl sesqui-2- | Sodium stearate | 8:0.75:0.75:0.5 | Hard opaque gel |
| | | ethylhexanoate/ethylene | | | |
| | | glycol | | | |
| 574 | d-limonene | Glyceryl sesqui-2- | Sodium stearate | 8:1:0.5:0.5 | Hard opaque gel |
| | | ethylhexanoate/ethylene | | | |
| | | glycol | | | |
| 575 | d-limonene | Ethox 4075/glycerin/ | Sodium stearate | 8:1:0.25:0.25:0.5 | Hard translucent gel |
| | | ethylene glycol | | | |
| 576 | d-limonene | Ethox 4075/glycerin/ | Sodium stearate | 8:1:0.25:0.25:0.5 | Hard translucent gel |
| | | ethylene glycol | | | |
| 577 | d-limonene | Ethox 4075/ethylene | Sodium stearate | 8.4:1:0.1:0.5 | Medium translucent gel |
| | | glycol | | | |
| 578 | d-limonene | Ethox 4074/ethylene | Sodium stearate | 8.4:1:0.1:0.5 | Medium opaque gel |
| | | glycol | | | |
| 579 | d-limonene | POP(200) isolignoceryl | | 8:1 | Clear liquid |
| | | alcohol | | | |
| 580 | d-limonene | POP(200) isolignoceryl | Sodium stearate | 8:1:0.5 | Opaque liquid |
| | | alcohol | | | |
| 581 | Di(ethylene | Di(propylene glycol) | Ethanol | 10:10:10 | Clear liquid |
| | glycol) | | | | |
| 582 | Water | Ethox 4075/ | Sodium lauryl sulfate | 83.4:2.4:2.8:11.4 | Thickened clear liquid |
| | | Cocamidopropyl betaine | | | Viscosity = 40,000 cps |
| 583 | Water | Ethox 4075/ | Ammonium lauryl | 78.7:2.0:1.5:2.8:15.0 | Thickened clear liquid |
| | | Cocamidopropyl betaine | sulfate/Ammonium | | Viscosity = 70,000 cps |
| | | | laureth sulfate | | |
| 584 | Water | Ethox 4075/ | Sodium laureth sulfate | 84.1:2.5:3.0:10.4 | Thickened opaque liquid |
| | | Cocamidopropyl betaine | | | Viscosity = 9,000 cps |
| 585 | Water | Ethox 4075/ | Sodium lauryl sulfate/ | 80.5:2.0:1.5:3.0:13.0 | Thickened opaque liquid |
| | | Cocamidopropyl betaine | Sodium laureth sulfate | | Viscosity = 28,000 cps |

*Drakeol 10 and Drakeol 7 are products of Penrico http://www.penreco.com/index.asp
**Dow Corning 1107 is a product of Dow Corning Silicones http://www.dowcorning.com/

Additional coupling agents that can be used with the invention include all those materials described in McCutcheon's publications published by The Manufacturing Confectioner Publishing Co. 175 Rock Road, Glen Rock, N.J. 07452 which titles include:

Emulsifiers and Detergents—North American Edition

Emulsifiers and Detergents—International Edition

Functional Materials—North American Edition

Functional Materials—International Edition

http://shop.gomc.com/Merchant2/merchant.mv?Screen=CTGY&Store_Code=TMC&Cat egory_Code=mccutch

Also, all surfactants described in CTFA International Buyers' Guide published by The Cosmetic, Toiletry, and Fragrance Association, 1101 17th Street, N.W., Suite 300, Washington, D.C. 20036-4702 (WWW.ctfa-buyersguide.org) can be used in the present invention.

The contents of all publications cited in this application are incorporated by reference in their entirety as if they were so denoted.

Although the present invention has been described with reference to specific details of certain embodiments thereof, it is not intended that such detail should be regarded as limitations upon the scope of the invention, except as and to the extent that they are included in the accompanying claims.

What is being claimed is:

1. A composition suitable for gelling solvents selected from the group consisting of polar solvents, non-polar solvents and combinations thereof comprising:
(a) an anionic component selected from the group consisting of a metallic soap, an anionic surfactant and mixtures thereof, and
(b) an enhancer comprising a coupling agent.

2. The composition of claim 1 wherein said anionic component is selected from the group consisting of ammonium lauryl sulfate, alkali metal lauryl sulfate, ammonium lauryl ether sulfate, alkali metal lauryl ether sulfate, alkali metal stearate, alkali metal salts, alkaline earth metal salts, ammonium, monoethanolamine, diethanolamine, tri-ethanolamine, or 2-amino-2-methylpropane-1,3-diol salt of alkyl sulfate having 8 to 22 carbon atoms in the alkyl group, polyoxyethylene (1 to 10 moles) alkyl ether sulfate having 8 to 22 carbon atoms in the alkyl group, polyoxyethylene (1 to 10 moles) alkylphenyl ether sulfate having 8 to 12 carbon atoms in the alkyl group, olefin sulfonate having 8 to 22 carbon atoms or alkylbenzene sulfonate having 9 to 18 carbon atoms in the alkyl group and mixtures thereof.

3. The composition of claim 2, wherein said metallic soap is sodium stearate.

4. The composition of claim 1, wherein said coupling agent is selected from the group consisting of:
(1) Monoester alkoxylates having the following chemical structure 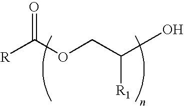
wherein R═H, C₁₋₂₅ primary alkyl, secondary alkyl, tertiary alkyl, branched alkyl, C₂-C₂₅ alkenyl, C₂-C₂₅ alkynyl, C₆-C₁₅ substituted alkylaryl, saturated and unsaturated C₅-C₁₀ cycloaliphatic wherein all Rs' may be optionally substituted with halogen or other functional groups selected from the group consisting of hydroxy, carboxy, sulfonate, aldehyde, ketone, R₁═H, C₁₋₂, phenyl and n=1-200;
(2) Monoamide derivatives having the following structural chemical formula 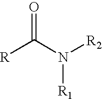
wherein R═H, C₁₋₂₅ primary alkyl, secondary alkyl, tertiary alkyl, branched alkyl, C₂-C₂₅ alkenyl, C₂-C₂₅ alkynyl, C₆-C₁₅ substituted alkylaryl, saturated and unsaturated C₅-C₁₀ cycloaliphatic wherein all Rs' may be optionally substituted with halogen or other functional groups selected from the group consisting of hydroxy, carboxy, sulfonate, aldehyde, ketone, R₁═H, C₁₋₂₀, (—CH₂CHR₃O—)ₙH (n=1-200) R₃═H, C₁₋₂, phenyl, R₂═H, C₁₋₂₀, —(CH₂CHR₃O—), (n=1-200) R₃═H, C₁₋₂, phenyl;
(3) Diamides having the following chemical structure 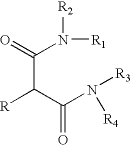
wherein R═H, C₁₋₂₅ primary alkyl, secondary alkyl, tertiary alkyl, branched alkyl, C₂-C₂₅ alkenyl, C₂-C₂₅ alkynyl, C₆-C₁₅ substituted alkylaryl, saturated and unsaturated C₅-C₁₀ cycloaliphatic wherein all Rs' may be optionally substituted with halogen or other functional groups selected from the group consisting of hydroxy, carboxy, sulfonate, aldehyde, ketone, R₁, R₂, R₃, and R₄═H, C₁₋₂₀, —(CH₂CHR₅O—)ₙH (n=1-200) R₅═H, C₁₋₂, phenyl, and R₁, R₂, R₃, and R₄ may all be the same or different at each designated position;
(4) Alcohol alkoxylates having the following chemical structure 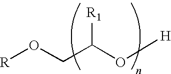
wherein R═H, C₁₋₂₅ primary alkyl, secondary alkyl, tertiary alkyl, branched alkyl, C₂-C₂₅ alkenyl, C₂-C₂₅ alkynyl, C₆-C₁₅ substituted alkylaryl, saturated and unsaturated C₅-C₁₀ cycloaliphatic wherein all Rs' may be optionally substituted with halogen or other functional groups selected from the group consisting of hydroxy, carboxy, sulfonate, aldehyde, ketone, R₁═H, C₁₋₂, Phenyl, n=1-200;
(5) Triglycerides (natural and synthetic) having the following chemical structure: 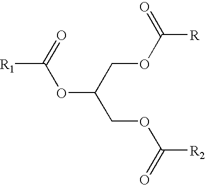
wherein R, R₁, and R₂═H, C₁₋₂₅ primary alkyl, secondary alkyl, tertiary alkyl, branched alkyl, C₂-C₂₅ alkenyl, C₂-C₂₅ alkynyl, C₆-C₁₅ substituted alkylaryl, saturated and unsaturated C₅-C₁₀ cycloaliphatic wherein all Rs' may be optionally substituted with halogen or other functional groups selected from the group consisting of hydroxy, carboxy, sulfonate, aldehyde, ketone, and may be the same or different at each position;
(6) Glycerides having the following chemical structure: 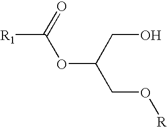
wherein R₁═H, C₁₋₂₅ primary alkyl, secondary alkyl, tertiary alkyl, branched alkyl, C₂-C₂₅ alkenyl, C₂-C₂₅ alkynyl, C₆-C₁₅ substituted alkylaryl, saturated and unsaturated C₅-C₁₀ cycloaliphatic wherein all R₁s' may be optionally substituted with halogen or other functional groups selected from the group consisting of hydroxy, carboxy, sulfonate, aldehyde, ketone, R═H or C₁₋₂₅ alkyl carbonyl, and may also be aromatic, unsaturated, saturated, branched, linear, or a cyclic hydrocarbon group having up to 25 carbon atoms;
(7) Sorbitan esters having the following chemical structure: 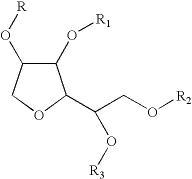
wherein each substituent R, R₁, R₂, and R₃ is independently selected from H, C₁-C₂₅ alkyl, alkylcarbonyl or alkenylcarbonyl, or —(CH₂CHR₄O—)ₙR′ (n=1-200), wherein R′ is selected from the group consisting of H, C₁-C₂₅ alkyl, alkylcarbonyl or alkenyl carbonyl, R₄═H, C₁₋₂, phenyl, R, R₁, R₂, and R₃ may be optionally substituted with halogen or other functional groups selected from the group consisting of hydroxy, carboxy, sulfonate, aldehyde, ketone, and R, R₁, R₂, and R₃ may be the same or different at each position;
(8) Polyglycerols having the following chemical formula: 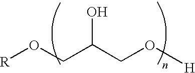
wherein R═H, C₁₋₂₅ alkyl, alkylcarbonyl or alkenylcarbonyl, R may be optionally substituted with halogen or other functional groups selected from the group consisting of hydroxy, carboxy, sulfonate, aldehyde, ketone, and n=2-25;
(9) Polyalkoxylates having the following chemical structure: 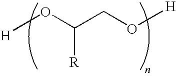
wherein R═H, C₁₋₂₅ primary alkyl, secondary alkyl, tertiary alkyl, branched alkyl, C₂-C₂₅ alkenyl, C₂-C₂₅ alkynyl, phenyl, C₆-C₁₅ substituted alkylaryl, saturated and unsaturated C₅-C₁₀ cycloaliphatic wherein all Rs' may be optionally substituted with halogen or other functional groups selected from the group consisting of hydroxy, carboxy, sulfonate, aldehyde, ketone and n=1-200;
(10) Ethoxylated triglycerides (castor oil derivatives) of the following structural formula 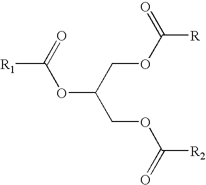
wherein R=alkoxylated ricinoleic residue, or alkoxylated 12-hydroxystearic acid residue and R, R₁, and R₂ may be the same or different at each position;
(11) Sorbitol alkoxylate esters of the following chemical formula: 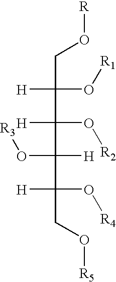
wherein each substituent R, R₁, R₂, R₃, R₄ and R₅ is independently selected from H, C₁-C₂₅ alkyl, alkylcarbonyl or alkenylcarbonyl, or —(CH₂CHR₄O—)ₙR′ (n=1-200), wherein R′ is selected from the group consisting of H, C₁-C₂₅ alkyl or alkenyl carbonyl, R₄═H, C₁₋₂, phenyl and R, R₁, R₂, and R₃ may be optionally substituted with halogen or other functional groups selected from the group consisting of hydroxy, carboxy, sulfonate, aldehyde, ketone, and R₁, R₂, R₃, R₄, and R₅ may be the same or different at each position;
(12) Sulfates having the following chemical formula: 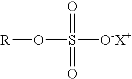
wherein R═H, C₁₋₂₅ alkyl or alkenyl, or —(CH₂CHR₄O—)ₙR′ (n=1-200), R′ is C₁-C₂₅ alkyl or alkenyl carbonyl, R₄═H, C₁₋₂, phenyl, and may be aromatic, unsaturated, saturated, branched, linear or cyclic group, X+=alkali or alkaline earth metal or ammonium;
(13) Polyamines having the following structural formula: 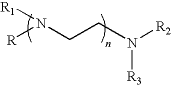
wherein R, R₁, R₂ and R₃ are selected from the group consisting of R═H, C₁₋₂₅, or —(CH₂CHR₄O—)ₙH (n=1-200), R₄═H, C₁₋₂ alkyl, phenyl, and may be aromatic, unsaturated, saturated, branched, linear or cyclic hydrocarbon group having up to 25 carbon atoms, and R, R₁, R₂, and R₃ may be the same or different and n=1-25;
(14) Citrate esters having the following chemical formula: 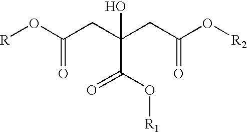
wherein R═H, C₁₋₂₅, or —(CH₂CHR₄O—)ₙR′ (n=1-200), R′ is C₁-C₂₅ alkyl or alkenyl carbonyl R₄↑H, C₁₋₂, phenyl, and may be aromatic, unsaturated, saturated, branched, linear or cyclic hydrocarbon group having up to 25 carbon atoms;
(15) Alkylene oxide esters having the following formula: 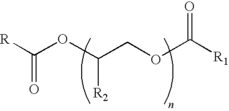
wherein R, R₁═H, C₁₋₂₅, and may be C₆-C₁₀ aromatic, or unsaturated, saturated, branched, linear or cyclic hydrocarbon groups having up to 25 carbon atoms, and R and R₁ may be the same or different, R₂═C₁₋₂, phenyl, n=1-200;
(16) Phosphates having the following formula: 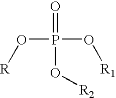
wherein R, R₁, and R₂═H, C₁₋₂₅, or —(CH₂CHR₃O—)ₙH (n=1-200) R₃═H, C₁₋₂, phenyl, and may also be aromatic, unsaturated, saturated, branched, linear, cyclic, alkyl alkoxylate, salt (metal, amine) and R, R₁, and R₂ may be the same or different. R, R₁ and R₂ may be derived from alkoxylated aliphatic alcohols or hydroxylated alkylaromatic;
(17) Tertiary amines having the formula: 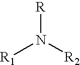
wherein R, R₁, and R₂═H, C₁₋₂₅, or —(CH₂CHR₃O—)ₙH (n=1-200) R₃═H, C₁₋₂, phenyl, and may be aromatic, unsaturated, saturated, branched, linear, or cyclic hydrocarbon groups having up to 25 carbon atoms. R, R₁, and R₂ may be the same or different;
(18) Ammonium quaternaries having the following formula: 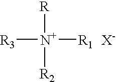
wherein R, R₁, R₂, and R₃═H, C₁₋₂₅, or —(CH₂CHR₃O—)ₙH (n=1-200) R₃═H, C₁₋₂, phenyl, and may be aromatic, unsaturated, saturated, branched, linear or cyclic aromatic groups having up to 25 carbon atoms. R, R₁, R₂, and R₃ may be the same or different, X=halide such a chloride or bromide or iodide or sulfate;
(19) Copolymers having the following chemical formula: 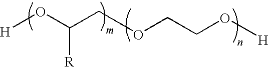
wherein R═H, C₁₋₂, phenyl, m=1-100 and n=1-100;
(20) Glycerol ester alkoxylates having the formula: 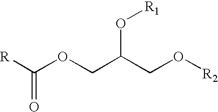
wherein R═H, C₁₋₂₅ alkyl, alkenyl or alkynyl, and may also be aromatic, unsaturated, saturated, branched, linear or cyclic hydrocarbon group having up to 25 carbon atoms, R₁═H, C₁₋₂₅ alkylcarbonyl, or —(CH₂CHR₃O—)ₙR′ (n=1-200), R′ may be C₁₋₂₅ alkylcarbonyl or C₂₋₂₅ alkenyl carbonyl, R₃═H, C₁₋₂, phenyl, when R₁=alkyl carbonyl, the group is C₁₋₂₅ and may also be aromatic, unsaturated, saturated, branched, linear, cyclic hydrocarbon groups having up to 25 carbon atoms, and R₂═R or R1 and may be the same or different;
(21) Alcohol alkoxylate esters of the formula: 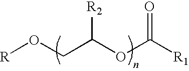
wherein R═H, C₁₋₂₅, and may be aromatic, unsaturated, saturated, branched, linear and cyclic, R₁═H, C₁₋₂₄, and may be aromatic, unsaturated, saturated, branched, linear and cyclic. R₂═H, C₁₋₂, or phenyl. R, R₁ and R₂ may be optionally substituted with halogen or other functional groups selected from the group consisting of hydroxy, carboxy, sulfonate, aldehyde, ketone; and
(22) Ester/amide phosphates of the following formula: 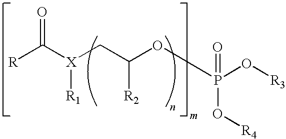
wherein X═N, O and when X=O, R₁ does not exist and when X═N, R₁ is the same as alkoxylate phosphate branch, R═H, C₁₋₂₅ and may be aromatic, unsaturated, saturated, branched, linear or cyclic. R₂═H, C₁₋₂, phenyl, (n=1-200). R₃═H, m, alkyl C₁₋₂₅, alkoxylate and R₄ is same or different than R₃.

5. The composition of claim 4 wherein said glyceryl ester is a glycerine 1.2 ester having 25% isostearic and 75% C₈-C₁₀ fatty acid moieties.

6. A composition suitable for gelling polar and non-polar solvents comprising:
(a) an anionic surfactant; and
(b) an enhancer selected from the group consisting of: isocetyl citrate, isoarachidyl citrate, POE(5) diTMP tetra-isostearate, trimethyl soya quaternary ammonium chloride, (C₁₂₋₁₈) dialkyldimethyl-ammonium chloride, soybean oil DEA transester, castor oil, POE(4) cetyl stearyl alcohol, POE(4) decyl alcohol, POE(6) decyl alcohol, POE(2) 2-ethylhexyl alcohol, Epal 1214 phosphate, C₈₋₁₀ triglyceride, CFA/DEA transamide TOFA salt, PPG 2025 TOFA ester, POE(2.5)cocoamine DES quaternary, stearic acid DEA amide, glycerine sesqui-cocoate, TOFA/DEA amide/soap, POE(3.3) CFA/MEA amide, DMSD/TEA DES quaternary, glycerine sesqui-C₈₋₁₀, POE(2) cocoamine, POE(5) castor oil, CFA/DEA transamide, PEG 600 dilaurate, PEG 200 dilaurate, PEG 400 dilaurate, PEG 600 ditallate, PEG 400 ditallate, POE(5) hydrogenated castor oil, POE(15) hydrogenated tallow amine DES quaternary, POE(4) laurate, POE(4) tallate, POE(2) tallow amine, POE(2) tallow amine DES quaternary, sorbitan monostearate (SMS), glycerine dilaurate, glycerine monosoyate, glycerine monostearate (GMS), hydrogenated castor oil (HCO), PEG 200, PEG 400, POE(1.1) laurate, POP(1.1) laurate, sorbitan monolaurate (SML), sorbitan monooleate (SMO), sorbitan monopalmitate (SMP), sorbitan trioleate (STO), soybean oil, triglycerol monostearate, decaglycerol monostearate, and tri-C₁₂₋₁₄ borate ester, 1,4-cyclohexanedimethanol, 1,6-hexane diol, 1-hexadecanaminium, n,n-dihexa-decyl-n-methyl-, chloride, 1H-imidazolium, 1-ethyl-2-(8-heptadecenyl)-4,5-dihydro-3-(2-hydroxyethyl)-, ethyl sulfate (salt), 2-ethyl-hexylamine, 2-phenoxyethanol, acylated polypeptides salt, benzyl-triethanolammonium chloride, C₁₂₋₁₈-dialkyldimethylammonium chloride, caprylic/capric 2-ethylhexanoate, caprylic/capric sesquicitrate, caprylic/capric sesquicitrate sodium salt, caprylic/capric sesquiglyceride, caprylic/capric triglyceride, caprylic/capric/isostearyl sesqui-glyceride, castor oil, cetylpyridinium chloride, coco imidazoline, coco-amine(2) dimethyl sulfate, coco-amine(2.5) diethyl sulfate, cocodimethylbenzylammonium chloride, coco ethyldimonium ethosulfate, cyclomethicone+octyl dimethicone ethoxy glucoside, decaglycerol mono-stearate, dendrimer surfactant, diethanol amine, diethanol amine/monoethanol amine octenyl succinate stearate, diethanol amine octenyl succinate stearate, diethanol amine stearate, diethanol amine tallate, diethanolamide isostearate, diethylenetriamine pentaacetic acid, dimethyl cetyl amine, dimethyl coco-amine benzyl chloride, dimethyl soya amine, dimethyl stearyl amine, dimethylaminopropylamine lauramide, dimethylsoyamine/triethanol amine diethyl sulfate, di-trimethylolpropane C₁₂₋₁₈ tetra ester, ditpnb azelate, dimethylamino-propylamine stearamide dimethyl sulfate, ethylene glycol, ethylene glycol mono-laurate, ethylene glycol monotungate, ethylene oxide propylene oxide copolymer, ethylenediamine tetra-acetic acid, glycerin, glycerol 2-ethylhexanoate, glycerol 2-ethylhexanoate sodium salt, glycerol benzoate, glycerol dilaurate, glycerol dimyristate, glycerol distearate, glycerol formal, glycerol monoisostearate, glycerol monosoyate, glycerol monostearate (gms), glycerol sesqui 2-ethylhexanoate, glycerol sesqui-cocoate, hydrogenated castor oil, isoarachidyl citrate, isocetyl citrate, isolignoceryl(200) behenate, lauric/dea transamide tofa salt, lauryl phosphate, lauryl(3.3) monoethanol amide, monoethanol amine, monoethanol amine octenyl succinate, monoethanol amine octenyl succinate stearate, oleoyl sarcosine, oleyl monoethanolamide phosphate, peg 200, peg 200 dilaurate, peg 400, peg 400 dilaurate, peg 400 ditallate, peg 600 dilaurate, peg 600 ditallate, peg 8000 diisostearate, poe(10) glycerol distearate, poe(10) glycerol formal, poe(12) glycerol dimyristate, poe(15) hydrogenated tallow amine des quat., poe(15) tallow diamine di-methyl sulfate, poe(2) 2-ethyl hexanol, poe(2) bisphenol A, poe(2) cocoamine, poe(2) tallow amine, poe(2) tallow amine diethyl sulfate, poe(2.2) dimerate, poe(200) isolignoceryl alcohol, poe(23) glycerol dimyristate, poe(23) glycerol dioleate, poe(23) lauryl alcohol, poe(25) behenyl alcohol, poe(25) hydrogenated castor oil isostearate, poe(3) lauryl alcohol, poe(3) tridecyl alcohol, poe(3) trimethylolpropane, poe(4) cetyl stearyl alcohol, poe(4) decyl alcohol, poe(4) laurate, poe(4) lauryl alcohol, poe(4) tallate, poe(5) castor oil, poe(5) di-trimethylolpropane tetra-isostearate, poe(5) hydrogenated castor oil, poe(5) hydrogenated castor oil oleate, poe(5) tridecyl phosphate potassium salt, poe(6) decyl alcohol, poe(7) decyl alcohol, poe(70) tristyrenated phenol sulfate, poe(9) isostearate, poly-12-hydroxystearate-dimethylaminopropylamine dimethyl sulfate, pop(2) cetyl alcohol, pop(2.2) dimerate, pop(200) isolignoceryl alcohol, pop(4)poe(1) cetyl alcohol, pop(5) laurate, pop(8) decyl alcohol, potassium dodecenylsuccinate, ppg 2025 tallate, ppg-460, propylene glycol monolaurate, propylene glycol monolaurate phosphate, propylene glycol monolaurate phosphate dea salt, propylene glycol monolaurate phosphate deta salt, propylene glycol monolaurate phosphate mea salt, propylene glycol monolaurate phosphate tea salt, sodium chloride, sodium laureth sulfate, sodium phosphate monobasic, sorbitan monolaurate (sml), sorbitan monooleate (smo), sorbitan monopalmitate (smp), sorbitan monostearate (sms), sorbitan trioleate (sto), soy lecithin, soybean oil, stearylamine(2) dimethyl sulfate, tallowpenta-methylpropylenediammonoim bismethosulfate, tridecyl phosphate, triethylmethyl-ammonium chloride, triglycerol monostearate, trilauryl borate, an trimethyl soya quaternary ammonium chloride.

7. A composition in gel form comprising:
(a) an anhydrous liquid carrier; and
(b) a gelling agent in an amount sufficient to impart gelation to the composition, the gelling agent being a mixture comprising an anionic surfactant and a glyceryl ester, wherein the composition is substantially anhydrous.

8. The composition according to claim 7 wherein the anhydrous liquid carrier is selected from the group consisting of non-volatile polysiloxanes, paraffinic hydrocarbon oils, and mixtures thereof.

9. The composition according to according to claim 8 wherein the non-volatile polysiloxanes are selected from the group consisting of polyalkylsiloxanes, polyarylsiloxanes, polyalkylarylsiloxanes, polyethersiloxane copolymers and mixtures thereof.

10. The composition according to claim 8, wherein the paraffinic hydrocarbon oil is selected from the group consisting of mineral oils, petrolatums, isodecanes, permethyls, isohexadecanes, isododecane and isoparaffins.

11. A composition in gel form comprising:
(a) a solvent selected from the group consisting of polar solvents, non-polar solvents and combinations thereof; and
(b) a gelling agent in an amount sufficient to impart gelation to the composition, the gelling agent being a mixture comprising an anionic surfactant and a coupling agent.

12. A cosmetic gel product on the basis of oils and gelling agents which product comprises 5 to 90% by weight of a liquid phase forming agent selected from among oils, mineral oils, hydrogenated hydrocarbons, alkenes, mono esters, di esters, tri esters and mixtures thereof, 0.1 to 20% by weight of a gelling agent comprising a mixture of an anionic surfactant and a coupling and all percentages being relative to the weight of the gel product, and wherein the gel product is transparent or a translucent solid stick.

13. A gel comprising:
(a) a gelling agent comprising a mixture of an anionic surfactant and a coupling agent; and (b) a liquid carrier.

14. A gel according to claim 13 in the form of a cosmetic composition.

15. A gel according to claim 14 wherein the cosmetic composition is in the form of a lipstick, gel, bar soap, lip balm, soft gel, cream, lotion, roll-on, makeup, facial moisturizer, or gel stick.

16. A gel according to claim 15 further comprising a moisturizer or protectant.

17. A gel according to claim 16 wherein the moisturizer or protectant is selected from the group consisting of aloe vera, allontoin, aluminum hydroxide gel, bismuth subnitrate, boric acid, calamine, cocoa butter, corn starch, dimethicone, glycerin, lanolin, kaolin, live yeast cell derivative, petrolatum, shark liver oil, sodium bicarbonate, sulfur, tannic acid, white petrolatum, zinc acetate, zinc carbonate and zinc oxide and mixtures thereof.

18. A process for preparing a gel, which comprises: homogeneously admixing a polar solvent, non-polar solvent or mixtures thereof with an effective gel forming amount of a composition comprising an anionic surfactant and a coupling agent; and allowing the mixture to stand until gel formation occurs.

19. The process of claim 18, wherein said homogeneous admixing is conducted at temperatures ranging from room temperature up to the boiling point of said non-polar organic liquid.
